(19) United States
(12) Patent Application Publication
(10) Pub. No.: US 2022/0161245 A1
(43) Pub. Date: May 26, 2022

(54) Compositions And Methods For Infrared-Light-Controlled Ruthenium-Catalyzed Olefin Metathesis

(71) Applicant: The Trustees of Columbia University in the City of New York, New York, NY (US)

(72) Inventors: Tomislav Rovis, Scarsdale, NY (US); David Cabanero, New York, NY (US)

(21) Appl. No.: 17/532,353

(22) Filed: Nov. 22, 2021

Related U.S. Application Data
(60) Provisional application No. 63/116,373, filed on Nov. 20, 2020.

(51) Int. Cl. B01J 31/22; C07C 67/035; C07D 207/48; C07D 223/04; C07D 309/18; C07C 41/32; B01J 35/00; B01J 31/18; C07C 6/06; C08G 61/08

(52) U.S. Cl. CPC B01J 31/2273; C08G 2261/418; C07D 207/48; C07D 223/04; C07D 309/18; C07C 41/32; B01J 35/004; B01J 31/1815; C07C 6/06; C08G 61/08; C07C 2601/10; B01J 2531/821; B01J 2231/543; B01J 2531/825; C08G 2261/228; C08G 2261/3325; C07C 67/035

(57) ABSTRACT

The present disclosure provides compositions and methods for metathesizing a first alkenyl or alkynyl group with a second alkenyl or alkynyl group, the composition comprising a ruthenium metathesis catalyst and a photo-redox catalyst that is activated by infrared light.

### RELATED APPLICATIONS

This application claims the benefit of priority of U.S. provisional application Ser. No. 63/116,373, filed Nov. 20, 2020, the entire contents of which are incorporated by reference herein.

### GOVERNMENT RIGHTS

This invention was made with government support under 2102672 awarded by the National Science Foundation. The government has certain rights in the invention.

### FIELD OF INVENTION

The present invention provides, inter alia, compositions and methods for controllable olefin metathesis.

### BACKGROUND OF THE INVENTION

Controlling polymer chain length and the average molecular weight dispersity is important for production of highly functional polymers at industrial scales. Ring-opening metathesis polymerization (ROMP) is an efficient polymerization method used to manufacture polymers with high fidelity and accuracy. The ring opening metathesis polymerization (ROMP) of alkenes is an important class of chain-growth polymerization that produces industrially important products. The driving force of these reactions is the relief of ring strain in cyclic olefins and a variety of heterogeneous and homogeneous catalysts have been developed and used in this context. ROMP is an attractive method to synthesize functional polymers as it is robust, produces linear materials with narrow molecular weight distributions and controlled average molecular weights.

Ring-closing metathesis (RCM) and cross-metathesis are also industrially important processes for production of compounds. However, ROMP, RCM and cross-metathesis are typically activated by metal catalysts that can make controlling chain length and molecular weight challenging. As such, there is an unmet need for methods and compositions that precisely tune catalysis of ROMP, RCM and cross-metathesis, which would enable improved compound production. The present disclosure is meant to address these limitations.

### SUMMARY OF THE INVENTION

In some embodiments, the disclosure provides compositions for metathesizing a first alkenyl or alkynyl group with a second alkenyl or alkynyl group, the composition comprising a ruthenium metathesis catalyst and a photo-redox catalyst that is activated by light in the deep-red region or the infrared region. In certain aspects, the deep-red light has a wavelength of about 600 nm to about 720 nm. In certain aspects, the infrared light has a wavelength of about 720 nm to about 2500 nm.

In other embodiments, the disclosure provides methods of spatially controlling a metathesis, comprising forming a mixture of a ruthenium metathesis catalyst, a photo-redox catalyst, and one or more compounds susceptible to metathesis; and applying deep-red light or infrared light to one or more regions of the mixture so as to give rise to one or more metathesized regions and one or more un-metathesized regions. In certain aspects, the deep-red light or infrared light is applied using a high resolution light source. In other aspects, at least one of the un-metathesized regions is covered with a photomask. In further aspects, the photomask is substantially opaque. In yet other aspects, the substrate is functionalized with the one or more compounds susceptible to metathesis.

The disclosed technology has application in a broad range of fields, including, e.g., polymer production, polymer patterning, photolithography and 3-D printing, among others. The disclosed technology presents numerous advantages over existing approaches, which advantages include, for example, minimal reagent requirements, the ability to use deep-red light or infrared light (thereby reducing or even eliminating the need for more complicated illumination sources), the ability to utilize a broad range of starting materials as to arrive at a broad range of products, and the ability to exert both temporal and spatial control over product formation.

### BRIEF DESCRIPTION OF THE DRAWINGS

The present application is further understood when read in conjunction with the appended drawings. For the purpose of illustrating the subject matter, exemplary embodiments of the subject matter are shown in these drawings; however, the presently disclosed subject matter is not limited to the specific methods, devices, and systems disclosed. In addition, the drawings are not necessarily drawn to scale. In the drawings:

FIG. 1 depicts temporal control of ROMP of cyclooctadiene through an in-situ LED-NMR On/Off study.

FIG. 2 depicts spatial control of synthesis of polydicyclopentadiene (pDCPD) to form leaf-like shapes.

FIG. 3 depicts spatial control of synthesis of polydicyclopentadiene (pDCPD) to form a circular shape.

FIG. 4 depicts spatial control of synthesis of polydicyclopentadiene (pDCPD) to form an X shape.

### DETAILED DESCRIPTION OF ILLUSTRATIVE EMBODIMENTS

The present invention may be understood more readily by reference to the following description taken in connection with the accompanying Figures and Examples, all of which form a part of this disclosure. It is to be understood that this invention is not limited to the specific products, methods, conditions or parameters described or shown herein, and that the terminology used herein is for the purpose of describing particular embodiments by way of example only and is not intended to be limiting of any claimed invention. Similarly, unless specifically otherwise stated, any description as to a possible mechanism or mode of action or reason for improvement is meant to be illustrative only, and the invention herein is not to be constrained by the correctness or incorrectness of any such suggested mechanism or mode of action or reason for improvement. Throughout this text, it is recognized that the descriptions refer to compositions and methods of making and using said compositions. That is, where the disclosure describes or claims a feature or embodiment associated with a composition or a method of making or using a composition, it is appreciated that such a description or claim is intended to extend these features or embodiment to embodiments in each of these contexts (i.e., compositions, methods of making, and methods of using).

In the present disclosure the singular forms “a,” “an,” and “the” include the plural reference, and reference to a particular numerical value includes at least that particular value, unless the context clearly indicates otherwise. Thus, for example, a reference to “a material” is a reference to at least one of such materials and equivalents thereof known to those skilled in the art, and so forth.

When a value is expressed as an approximation by use of the descriptor “about,” it will be understood that the particular value forms another embodiment. In general, use of the term “about” indicates approximations that can vary depending on the desired properties sought to be obtained by the disclosed subject matter and is to be interpreted in the specific context in which it is used, based on its function. The person skilled in the art will be able to interpret this as a matter of routine. In some cases, the number of significant figures used for a particular value may be one non-limiting method of determining the extent of the word “about.” In other cases, the gradations used in a series of values may be used to determine the intended range available to the term “about” for each value. Where present, all ranges are inclusive and combinable. That is, references to values stated in ranges include every value within that range.

It is to be appreciated that certain features of the invention which are, for clarity, described herein in the context of separate embodiments, may also be provided in combination in a single embodiment. That is, unless obviously incompatible or specifically excluded, each individual embodiment is deemed to be combinable with any other embodiment(s) and such the combination is considered to be another embodiment. Conversely, various features of the invention that are, for brevity, described in the context of a single embodiment, may also be provided separately or in any sub-combination. Finally, while an embodiment may be described as part of a series of steps or part of a more general structure, each said step may also be considered an independent embodiment in itself, combinable with others.

The transitional terms “comprising,” “consisting essentially of,” and “consisting” are intended to connote their generally in accepted meanings in the patent vernacular; that is, (i) “comprising,” which is synonymous with “including,” “containing,” or “characterized by,” is inclusive or open-ended and does not exclude additional, unrecited elements or method steps; (ii) “consisting of” excludes any element, step, or ingredient not specified in the claim; and (iii) “consisting essentially of” limits the scope of a claim to the specified materials or steps “and those that do not materially affect the basic and novel characteristic(s)” of the claimed invention. Embodiments described in terms of the phrase “comprising” (or its equivalents), also provide, as embodiments, those which are independently described in terms of “consisting of” and “consisting essentially of.” For those embodiments provided in terms of “consisting essentially of” the basic and novel characteristic(s) is the facile operability of the methods (and the systems used in such methods and the compositions derived therefrom) to prepare and use the inventive materials, and the materials themselves, where the methods and materials are capable of delivering the highlighted properties using only the elements provided in the claims. That is, while other materials may also be present in the inventive compositions, the presence of these extra materials is not necessary to provide the described benefits of those compositions or devices (i.e., the effects may be additive) and/or these additional materials do not compromise the performance of the product compositions or devices. Similarly, where additional steps may also be employed in the methods, their presence is not necessary to achieve the described effects or benefits and/or they do not compromise the stated effect or benefit.

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.” This includes, without limitation, that a genus presenting multiple parameters, each parameter presenting multiple options, represents that collection of individual embodiments including any and every combination of these variables and options. By means of illustration only, a composition described in terms of two variables A and B, each variable presenting two options (a) and (b), includes, as independent embodiments, the subgenera A(a)-B(a), A(a)-B(b), A(b)-B(a), and A(b)-B(b). This principle can be applied to larger numbers of variables and options, such that any one or more of these variable or options can be independently claimed or excluded. Likewise, a definition such as C₁₋₃-alkyl includes C₁-alkyl, C₂-alkyl, C₃-alkyl, C₁₋₂-alkyl, C₂₋₃-alkyl, and C₁₋₃-alkyl as separate embodiments.

Because each individual element of a list, and every combination of that list, is a separate embodiment, it should be apparent that any description of a genus or subgenus also included those embodiments where one or more of the elements are excluded, without the need for the disclosure of the exclusion. For example, a genus described as containing elements “A, B, C, D, E, or F” also includes the embodiments excluding one or more of these elements, for example “A, C, D, E, or F;” “A, B, D, E, or F;” “A, B, C, E, or F;” “A, B, C, D, or F;” “A, B, C, D, or E;” “A, D, E, or F;” “A, B, C, or F;” “A, E, or F;” “A, C, E, or F;” “A or F;” etc., without the need to explicitly delineate the exclusions.

The term “alkyl” as used herein refers to a linear, branched, or cyclic saturated hydrocarbon group typically although not necessarily containing 1 to about 30 carbon atoms, in some cases, from 1 to about 12 carbon atoms, such as methyl, ethyl, n-propyl, isopropyl, n-butyl, isobutyl, t-butyl, octyl, decyl, and the like, as well as cycloalkyl groups such as cyclopentyl, cyclohexyl and the like, or in other cases from about 12 to about 24 or 30 carbon atoms (e.g., oleic and other fatty or saturated acids). Generally, although again not necessarily, alkyl groups herein can also contain 1 to about 12 carbon atoms or 1 to 6 carbon atoms. The term “cycloalkyl” intends a cyclic alkyl group, typically having 4 to 8, preferably 5 to 7, carbon atoms. The term “substituted alkyl” refers to alkyl groups substituted with one or more substituent groups, and the terms “heteroatom-containing alkyl” and “heteroalkyl” refer to alkyl groups in which at least one carbon atom is replaced with a heteroatom. If not otherwise indicated, the terms “alkyl” and “lower alkyl” include linear, branched, cyclic, unsubstituted, substituted, and/or heteroatom-containing alkyl and lower alkyl groups, respectively.

The term “haloalkyl” as used herein refers to an alkyl groups as described, wherein one or more hydrogen atom is replaced with a halo. In some embodiments, haloalkyl includes an alkyl group substituted with one or more F. In some embodiments, haloalkyl includes an alkyl group substituted with one or more Br. In some embodiments, haloalkyl includes an alkyl group substituted with one or more Cl. In some embodiments, haloalkyl includes an alkyl group substituted with one or more I. Examples of haloalkyl groups include fluorinated alkyl groups including, without limitation, CF₃, CF₂H, CFH₂, CH₂CF₃, CH₂CH₂CF₃, CH₂CH₂CH₂CF₃, and CH₂CH₂CH₂CH₂CF₃.

The term “alkenyl” as used herein refers to a linear, branched, or cyclic hydrocarbon group of 2 to about 30 carbon atoms containing at least one double bond, such as ethenyl, n-propenyl, isopropenyl, n-butenyl, isobutenyl, octenyl, decenyl, tetradecenyl, hexadecenyl, eicosenyl, tetracosenyl, and the like. In some embodiments, alkenyl groups contain 2 to about 12 carbon atoms, preferably 2 to about 6 carbon atoms. “Alkenyl” also includes vinyl groups, wherein the double bond is at a terminal location of the molecule. The term “substituted alkenyl” refers to alkenyl groups substituted with one or more substituent groups. If not otherwise indicated, the term “alkenyl” includes linear, branched, cyclic, unsubstituted, and/or substituted alkenyl groups, respectively.

The term “alkynyl” as used herein refers to a linear, branched, or cyclic hydrocarbon group of 2 to about 30 carbon atoms containing at least one triple bond, such as ethynyl, n-propynyl, isopropynyl, n-butynyl, isobutynyl, octynyl, decynyl, tetradecynyl, hexadecynyl, eicosenyl, tetracosenyl, and the like. In some embodiments, alkynyl groups contain 2 to about 12 carbon atoms, preferably 2 to about 6 carbon atoms. The term “substituted alkynyl” refers to alkynyl groups substituted with one or more substituent groups. If not otherwise indicated, the term “alkynyl” includes linear, branched, cyclic, unsubstituted, and/or substituted alkynyl groups, respectively.

The term “alkoxy” as used herein intends an alkyl group bound through a single, terminal ether linkage; that is, an “alkoxy” group may be represented as —O-alkyl where alkyl is as defined above. An “alkoxy” group includes an alkoxy group containing 1 to 6 carbon atoms, i.e., methoxy, ethoxy, propoxy, butoxy, pentoxy, or hexoxy.

The term “aryl” as used herein, and unless otherwise specified, refers to an aromatic substituent or structure containing a single aromatic ring or multiple aromatic rings that are fused together, directly linked, or indirectly linked (such that the different aromatic rings are bound to a common group such as a methylene or ethylene moiety). In some embodiments, the aryl ring is unfused. In other embodiments, the aryl is a fused aryl. In further embodiments, the aryl is a bridged aryl. Unless otherwise modified, the term “aryl” refers to carbocyclic structures. Preferred aryl groups contain 5 to 24 carbon atoms, and particularly preferred aryl groups contain 5 to 14 carbon atoms. Exemplary aryl groups contain one aromatic ring or two fused or linked aromatic rings, e.g., phenyl, naphthyl, biphenyl, diphenylether, diphenylamine, benzophenone, and the like. “Substituted aryl” refers to an aryl moiety substituted with one or more substituent groups.

The term “acyl” refers to substituents having the formula —(CO)-alkyl (alkylcarbonyl), —(CO)-aryl (arylcarbonyl), or —(CO)-aralkyl, and the term “acyloxy” refers to substituents having the formula —O(CO)-alkyl, —O(CO)-aryl, or —O(CO)-aralkyl, wherein “alkyl,” “aryl, and “aralkyl” are as defined above.

The terms “halo,” “halide,” and “halogen” are used in the conventional sense to refer to a chloro, bromo, fluoro, or iodo substituent.

The term “heteroatom-containing” refers to a hydrocarbon molecule or a molecular fragment in which one or more carbon atoms is replaced with an atom other than carbon, e.g., nitrogen, oxygen, sulfur, phosphorus or silicon, typically nitrogen, oxygen or sulfur. Similarly, the term “heteroalkyl” refers to an alkyl substituent that is heteroatom-containing, the term “heterocyclic” refers to a cyclic substituent that is heteroatom-containing, the terms “heteroaryl” and heteroaromatic” respectively refer to “aryl” and “aromatic” substituents that are heteroatom-containing, and the like. It should be noted that a “heterocyclic” group or compound may or may not be aromatic, and further that “heterocycles” may be monocyclic, bicyclic, or polycyclic as described above with respect to the term “aryl.”

Non-limiting examples of heteroaryl groups include azepinyl, acridinyl, carbazolyl, cinnolinyl, furanyl, furazanyl, furanonyl, isothiazolyl, imidazolyl, indazolyl, indolyl, isoindolyl, indolinyl, isoindolinyl, isoquinolyl, indolizinyl, isoxazolyl, naphthyridinyl, oxadiazolyl, oxazolyl, oxiranyl, phenazinyl, phenothiazinyl, phenoxazinyl, phthalazinyl, pteridinyl, purinyl, pyranyl, pyrrolyl, pyrrolidinyl, pyrazolyl, pyridinyl, pyrazinyl, pyrimidinyl, pyridazinyl, quinazolinyl, quinoxalinyl, quinolinyl, isoquinolinyl, tetrahydroquinolinyl, thiazolyl, thiadiazolyl, thiopyranyl, triazolyl, tetrazolyl, triazinyl, and thiophenyl. Non-limiting examples of heteroatom-containing alicyclic groups are pyrrolidino, morpholino, piperazino, and piperidino.

The term “aryloxy” as used herein refers to a O-aryl group, wherein the aryl group is defined herein.

The term “aralkyl” refers to an alkyl group with an aryl substituent (-alkyl-aryl), wherein aryl and alkyl are defined herein. Similarly, the term “aralkyloxy” refers to an alkyl group with a O-aryl group -alkyl-O-aryl), wherein aryl and alkyl are defined herein.

The term “acyloxy” as used herein refers to an —O-acyl, wherein acyl is defined herein. The term “acyloxy” includes groups such as alkylcarbonyloxy (—OC(O)alkyl), arylcarbonyloxy (—OC(O)aryl), alkoxycarbonyl (—C(O)Oalkyl), and aryloxycarbonyl (—C(O)Oaryl), wherein the alkyl, aryl, and alkoxy groups are defined herein.

The term “halocarbonyl” as used herein refers to the —C(O)—X, wherein X is a halo group defined herein.

The term “carboxy” refers to a —C(O)OH, carboxylato or “carboxyl” refers to a —C(O)O—, and carbamoyl refers to a —C(O)NH₂ group.

The terms mono-(alkyl)-substituted carbamoyl and di-(alkyl)-substituted carbamoyl refers to —C(O)NH(alkyl) and —C(O)N(alkyl)₂ groups, respectively, wherein the alkyl groups are defined herein and independently chosen.

The terms “mono-(aryl)-substituted carbamoyl”, di-(aryl)substituted carbamoyl,” and “, di-N-(alkyl),N-(aryl)-substituted carbamoyl” refer to —C(O)NH-aryl, —C(O)N(aryl)₂), and —C(O)N(aryl)(alkyl) groups, wherein alkyl and aryl are defined herein and independently chosen.

The term “thiocarbamoyl” refers to the —C(S)NH₂ group. Similarly, the terms mono-(alkyl)-substituted thiocarbamoyl refers to the —C(S)NH(alkyl)) and the di-(alkyl)-substituted thiocarbamoyl refers to the —C(S)N(alkyl)₂), wherein the alkyl group is defined herein and is independently selected.

The terms “mono-(aryl)substituted thiocarbamoyl” and “di-(C₅₋₂₄aryl)-substituted thiocarbamoyl” refers to —C(S)NH-aryl and —C(S)N(C₅₋₂₄aryl)₂ groups, wherein the aryl groups are defined herein and are independently chosen.

The term “amino” refers to the NH₂ group. Similarly, alkyl-substituted amino groups include “mono-(alkyl)-substituted amino” and di-(alkyl)-substituted amino, wherein the alkyl groups are defined herein and independently chosen. Further, aryl-substituted amino groups include mono-(aryl)substituted amino and di-(aryl)-substituted amino, wherein the aryl groups are defined herein and independently chosen.

The term “amido” refers to the —NHC(O)— group, and includes alkylamido and arylamido groups. The terms “alkylamido” and “arylamido” refers to —NHC(O)alkyl and —NHC(O)aryl, wherein alkyl and aryl are defined herein.

The terms “alkylthio” and “arylthiol” as used herein refer to —S-alkyl and —S-aryl, groups, respectively, wherein alkyl and aryl are defined herein.

The term “sulfonyl” refers to the SO₂ group.

The term “germyl” refers to a GeR^{Z}₃ group, the term “stannyl” refers to a SnR^{Z}₃ group, the term “boryl” refers to a BH₂, BH(R^{Z}), B(R^{Z})₂, or B(OR^{Z})₂, group, and “silyl” refers to a SiR^{Z}₃, wherein R^{Z} is, independently, in each instance C₁₋₁₂alkyl or aryl as defined herein.

By “substituted” as in “substituted alkyl,” “substituted aryl,” and the like, as alluded to in some of the aforementioned definitions, is meant that in the alkyl, aryl, heteroaryl, or other moiety, at least one hydrogen atom bound to a carbon (or other) atom is replaced with one or more non-hydrogen substituents. Examples of such substituents include, without limitation, halo (e.g., F, Cl, Br, I), OH, sulfhydryl, alkoxy, aryloxy, aralkyloxy, acyl (including alkylcarbonyl, arylcarbonyl, acyloxy (alkylcarbonyloxy and arylcarbonyloxy), alkoxycarbonyl, aryloxycarbonyl, halocarbonyl, carboxy, carboxylate), carbamoyl, mono-(alkyl)-substituted carbamoyl, di-(alkyl)-substituted carbamoyl, mono-(aryl)-substituted carbamoyl, di-(aryl)substituted, di-N-(alkyl),N-(aryl)-substituted carbamoyl, thiocarbamoyl, mono-(alkyl)-substituted thiocarbamoyl, di-(alkyl)-substituted thiocarbamoyl, mono-(aryl)substituted thiocarbamoyl, di-(aryl)-substituted thiocarbamoyl, di-N-(alkyl), N-(aryl)-substituted thiocarbamoyl, CN, OCN, thiocyanato, formyl, C(S)H, NH₂, mono-(alkyl)-substituted amino, di-(alkyl)-substituted amino, mono-(aryl)substituted amino, di-(aryl)-substituted amino, alkylamido, arylamido, NO₂, NO, alkylthio (—S-alkyl), arylthio (—S-aryl), alkyl, alkenyl, alkynyl, aryl, and aralkyl. Within these substituent structures, the “alkyl,” “alkylene,” “alkenyl,” “alkenylene,” “alkoxy,” “aromatic,” “aryl,” “aryloxy,” and “aralkyl” moieties may be optionally fluorinated or perfluorinated.

“Optional” or “optionally” means that the subsequently described circumstance may or may not occur, so that the description includes instances where the circumstance occurs and instances where it does not. For example, the phrase “optionally substituted” means that a non-hydrogen substituent may or may not be present on a given atom, and, thus, the description includes structures wherein a non-hydrogen substituent is present and structures wherein a non-hydrogen substituent is not present.

### Compositions

The present disclosure is related to compositions and methods for metathesizing unsaturated compounds using one or more of a ruthenium catalyst, a photo-redox catalyst, and deep-red light or infrared light, such as near infrared (NIR) light. In some embodiments, it is believed that the mode of activation is the energy or electron transfer from/to the excited state of the photocatalyst that induces ligand dissociation and thus starts metathesis. In other embodiments, upon irradiation by deep-red light or infrared light, the photocatalyst is excited by absorption of a photon. In further embodiments, it also is believed that the methods induce dissociation of one of the carbene ligands from the latent metathesis catalyst, thus generating the active species that can start to promote ROMP or its mechanistically related congeners. In yet other embodiments, turning the light off leads to re-coordination of the carbene ligand onto the ruthenium catalyst and therefore is thought to shut down metathesis since the active catalyst is no longer present in the media. As a consequence, in some embodiments, the methods permit controlling initiation of the ROMP and accurately controlling the length of the polymer chains by modulating the irradiation time—these features that cannot be achieved with current systems in ROMP, and further expands the scope and ease of production of industrially relevant materials.

According to some embodiments, this disclosure relies on the activation of an external photocatalyst in order to activate the ruthenium metathesis catalyst and therefore start the polymerization event via an on and off process. In other embodiments, this disclosure provides a photocatalyst that is only catalytically-active upon absorption of deep-red light or infrared light, providing an external handle for precisely controlling initiation and termination of ROMP. As such, in further embodiments, this disclosure provides a switchable photocatalyst for the industrial fabrication of polymers with controlled weights, chain length, and dispersity. In yet other embodiments, the photo-redox promoted ring opening metathesis polymerization provides increased control over initiation and should serve as a tool that enables to precisely control the length of the polymer chains, and thus the properties of the polymer, by controlling the irradiation time.

Accordingly, one embodiment of the present disclosure is a composition for olefin metathesis comprising a latent metathesis catalyst, and a photocatalyst, wherein the olefin metathesis is controlled by deep-red light or infrared light irradiation. Thus, in some embodiments, the present disclosure provides the use of a latent ruthenium metathesis catalyst bearing two poorly dissociable carbene ligands that is not active for metathesis at ambient temperature without external activation.

As used herein, an “olefin metathesis” is an organic reaction that entails the redistribution of fragments of alkenes (olefins) by the scission and regeneration of carbon-carbon double bonds. As used herein, a “latent catalyst” is a catalyst that can be “switched on” from an inactive state by application of an external trigger such as, e.g., heat or light.

In other embodiments of the present disclosure, compositions for alkyne metathesis are provided comprising a latent metathesis catalyst, and a photocatalyst, wherein the alkyne metathesis is controlled by deep-red light or infrared light irradiation.

In further embodiments of the present disclosure, compositions for mixed olefin/alkyne metathesis are provided comprising a latent metathesis catalyst, and a photocatalyst, wherein the olefin/alkyne metathesis is controlled by deep-red light or infrared light irradiation.

The metathesis described herein is performed using a ruthenium metathesis catalyst and a photo-redox catalyst that is activated by light. The terms “photocatalyst” and “photo-redox catalyst” are interchangeable and refer to a catalyst that is activated by light. In some embodiments, the light is deep-red light or infrared light. In some embodiments, the light is deep-red light. In some embodiments, the light is infrared light. In some embodiments, the infrared light is near infrared (NIR) light.

“Deep-red” light as used herein refers to light that has a wavelength of about 600 nm to about 720 nm. In some embodiments, the deep-red light has a wavelength of about 610 nm to about 710 nm. In some embodiments, the deep-red light has a wavelength of about 620 nm to about 700 nm. In some embodiments, the deep-red light has a wavelength of about 630 nm to about 690 nm. In some embodiments, the deep-red light has a wavelength of about 640 nm to about 680 nm. In some embodiments, the deep-red light has a wavelength of about 650 nm to about 670 nm. In some embodiments, the deep-red light has a wavelength of about 660 nm.

In some embodiments, the deep-red light has a wavelength of about 600 nm to about 610 nm. In some embodiments, the deep-red light has a wavelength of about 610 nm to about 620 nm. In some embodiments, the deep-red light has a wavelength of about 620 nm to about 630 nm. In some embodiments, the deep-red light has a wavelength of about 630 nm to about 640 nm. In some embodiments, the deep-red light has a wavelength of about 640 nm to about 650 nm. In some embodiments, the deep-red light has a wavelength of about 650 nm to about 660 nm. In some embodiments, the deep-red light has a wavelength of about 660 nm to about 670 nm. In some embodiments, the deep-red light has a wavelength of about 670 nm to about 680 nm. In some embodiments, the deep-red light has a wavelength of about 680 nm to about 690 nm. In some embodiments, the deep-red light has a wavelength of about 690 nm to about 700 nm. In some embodiments, the deep-red light has a wavelength of about 700 nm to about 710 nm. In some embodiments, the deep-red light has a wavelength of about 710 nm to about 720 nm.

“NIR light” as used herein refers to light that has a wavelength of about 720 nm to about 2500 nm. In some embodiments, the NIR light has a wavelength of about 720 to about 2000 nm. In some embodiments, the NIR light has a wavelength of about 720 to about 1500 nm. In some embodiments, the NIR light has a wavelength of about 720 to about 1000 nm. In some embodiments, the NIR light has a wavelength of about 720 to about 900 nm. In some embodiments, the NIR light has a wavelength of about 720 to about 800 nm. In some embodiments, the NIR light has a wavelength of about 720 to about 780 nm. In some embodiments, the NIR light has a wavelength of about 730 to about 750 nm. In some embodiments, the NIR light has a wavelength of about 740 nm.

In some embodiments, the NIR light has a wavelength of about 720 nm to about 730 nm. In some embodiments, the NIR light has a wavelength of about 730 nm to about 740 nm. In some embodiments, the NIR light has a wavelength of about 740 nm to about 750 nm. In some embodiments, the NIR light has a wavelength of about 750 nm to about 760 nm. In some embodiments, the NIR light has a wavelength of about 760 nm to about 770 nm. In some embodiments, the NIR light has a wavelength of about 770 nm to about 780 nm. In some embodiments, the NIR light has a wavelength of about 780 nm to about 790 nm. In some embodiments, the NIR light has a wavelength of about 790 nm to about 800 nm. In some embodiments, the NIR light has a wavelength of about 800 nm to about 810 nm. In some embodiments, the NIR light has a wavelength of about 810 nm to about 820 nm. In some embodiments, the NIR light has a wavelength of about 820 nm to about 830 nm. In some embodiments, the NIR light has a wavelength of about 830 nm to about 840 nm. In some embodiments, the NIR light has a wavelength of about 840 nm to about 850 nm. In some embodiments, the NIR light has a wavelength of about 850 nm to about 860 nm. In some embodiments, the NIR light has a wavelength of about 860 nm to about 870 nm. In some embodiments, the NIR light has a wavelength of about 870 nm to about 880 nm. In some embodiments, the NIR light has a wavelength of about 880 nm to about 890 nm. In some embodiments, the NIR light has a wavelength of about 890 nm to about 900 nm.

The term “activated by infrared or NIR light” or “activated by deep-red light” refers to the state of photo-redox catalyst going from unreactive to reactive.

The inventors determined that photo-redox catalysts that are highly oxidizing contribute to the ease of metathesis. Thus, in some embodiments, the photo-redox catalyst has an oxidizing potential of about 1.5 to about 3 volts, i.e., “highly oxidizing”. In further embodiments, the oxidizing potential of the photo-redox catalyst is about 1.5 to about 2.75 volts, about 1.5 to about 2.5 volts, about 1.5 to about 2 volts, about 1.75 to about 3 volts, about 1.75 to about 2.75 volts, about 1.75 to about 2.5 volts, about 1.75 to about 2 volts, about 2 to about 3 volts, or about 2 to about 2.5 volts. In other embodiments, the oxidizing potential is about 1.5 volts, 1.6 volts, 1.7 volts, 1.75 volts, 1.8 volts, 1.9 volts, 2 volts, 2.1 volts, 2.2 volts, 2.3 volts, 2.4 volts, 2.5 volts, 2.6 volts, 2.7 volts, 2.8 volts, 2.9 volts, or about 3 volts.

In some embodiments, the photo-redox catalyst comprises Osmium.

In some embodiments, the photo-redox catalyst is Os(phen)₃(PF₆)₂; wherein phen is 1,10 phenanthroline.

In some embodiments, the photo-redox catalyst is

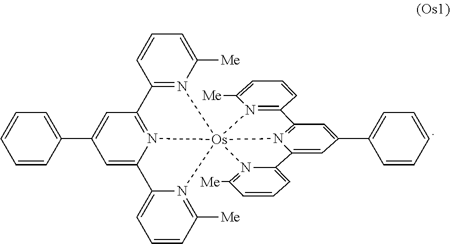

In some embodiments, the photo-redox catalyst is

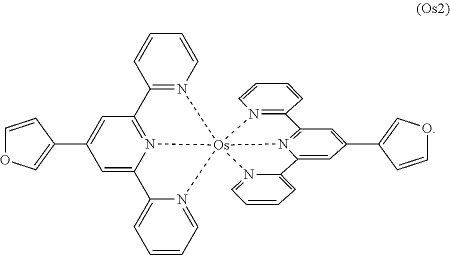

In some embodiments, the photo-redox catalyst is

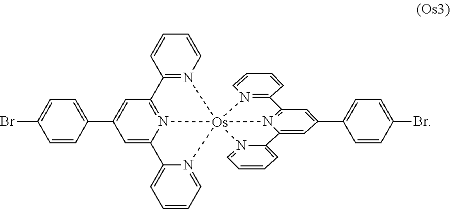

In some embodiments, the photo-redox catalyst is

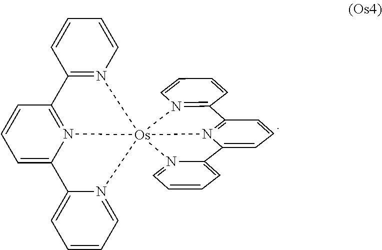

In some embodiments, the photo-redox catalyst is

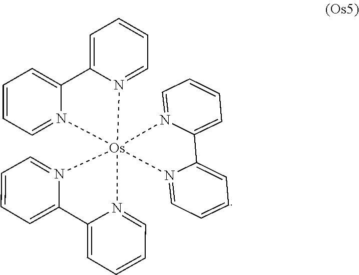

The metathesis of the disclosure also comprises the inclusion of a ruthenium catalyst. In some embodiments, the ruthenium catalyst is a latent ruthenium catalyst. The term “latent” as used herein refers to the state of the catalyst when it is inactivated, i.e., inactive or extremely sluggish. In some aspects, it is believed that the latent form of the ruthenium catalyst converts to an activated or active version of the ruthenium catalyst upon loss of one or more of its ligands. In some aspects, this loss of ligand is believed to arise by interaction of the Ru catalyst with the excited state of the photocatalyst leading to ligand oxidation and dissociation from Ru. In some embodiments, the ruthenium catalyst of Formula (I) or Formula II:

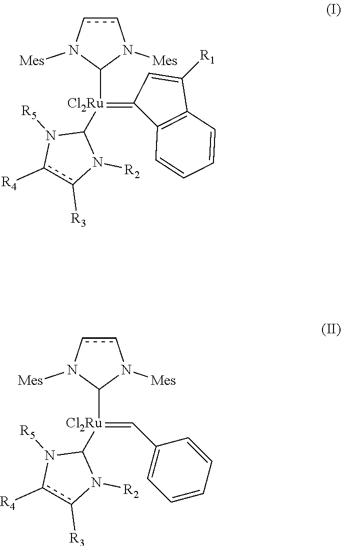

wherein:

R¹ is H, C₁₋₆alkyl, C₂₋₆alkenyl, aryl, or heteroaryl;

R² to R⁵ are, independently, H, C₁₋₆alkyl, C₁₋₆alkoxy, C₃₋₈ cycloalkyl, halo, or aryl; and

Mes is

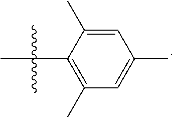 [SMILES: *c1c(C)cc(C)cc1C]

In the structure of Formula (I) or Formula (II), R¹ is H, C₁₋₆alkyl, C₂₋₆alkenyl, aryl, or heteroaryl. In some embodiments, R¹ is H. In further embodiments, R¹ is C₁₋₆alkyl, such as methyl, ethyl, propyl, butyl, pentyl, or hexyl. In other embodiments, R¹ is C₂₋₆alkenyl, such as ethenyl, propenyl, butenyl, pentenyl, or hexenyl. In further embodiments, R¹ is aryl such as a C₅₋₂₄ aryl, more preferably C₅₋₁₄ aryl. In other embodiments, the aryl is an optionally substituted phenyl, naphthyl, or biphenyl. In yet further embodiments, R¹ is phenyl. In yet other embodiments, R¹ is heteroaryl such as azepinyl, acridinyl, carbazolyl, cinnolinyl, furanyl, furazanyl, furanonyl, isothiazolyl, imidazolyl, indazolyl, indolyl, isoindolyl, indolinyl, isoindolinyl, isoquinolyl, indolizinyl, isoxazolyl, naphthyridinyl, oxadiazolyl, oxazolyl, oxiranyl, phenazinyl, phenothiazinyl, phenoxazinyl, phthalazinyl, pteridinyl, purinyl, pyranyl, pyrrolyl, pyrrolidinyl, pyrazolyl, pyridinyl, pyrazinyl, pyrimidinyl, pyridazinyl, quinazolinyl, quinoxalinyl, quinolinyl, isoquinolinyl, tetrahydroquinolinyl, thiazolyl, thiadiazolyl, thiapyranyl, triazolyl, tetrazolyl, triazinyl, or thiophenyl.

R² to R⁵ are, independently in each occurrence, H, C₁₋₆alkyl, C₁₋₆alkoxy, halo, or aryl. In some embodiments, R² to R⁵ are H. In further embodiments, R² to R⁵ are, independently in each occurrence, H or C₁₋₆alkyl. In other embodiments, R² to R⁵ are, independently in each occurrence, H or C₁₋₆alkoxy. In further embodiments, R² to R⁵ are, independently in each occurrence, H or halo. In yet other embodiments, R² to R⁵ are, independently in each occurrence, H or aryl.

In other embodiments, the ruthenium catalyst is:

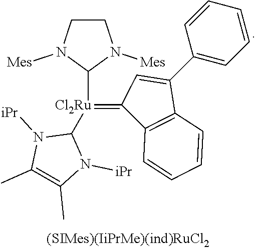

In other embodiments, the ruthenium catalyst is:

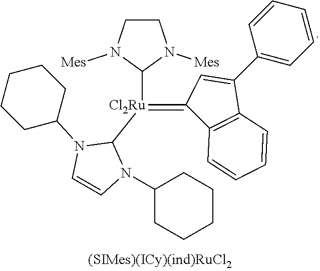

In other embodiments, the ruthenium catalyst is:

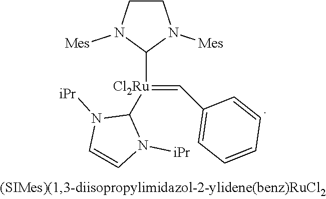

A scope of the metathesis described herein is not limited to those disclosed herein. Instead, one of skill in the art would be able to select suitable compounds to utilize in the metathesis described herein. In general, the compounds that may be metathesized according to the disclosure contain at least one point of unsaturation. The term “unsaturation” as used herein refers to a double or triple bond or any combination thereof. The term “double bond” as noted herein refers to a C═C group and a “triple bond” refers to a C≡C bond, either of which being contained in a chemical compound. The chemical compound containing a double bond is known in the art as an “alkene” or “olefin,” which terms may be used interchangeably. Thus, a substituent on a molecule having a double bond is an “alkenyl” group. Similarly, a chemical compound containing a triple bond is known in the art as an “alkyne.” Thus, a substituent on a molecule having a triple bond is an “alkynyl” group. In some embodiments, the metathesis is performed on a compound having two points of unsaturation. In other embodiments, the metathesis is performed on a first compound having at least one point of unsaturation and a second compound having at least one point of unsaturation.

As such, the present disclosure is directed to metathesizing a first alkenyl or alkynyl group with a second alkenyl or alkynyl group. In some embodiments, the present disclosure provides metathesizing a first alkenyl group with a second alkenyl group. In other embodiments, the present disclosure provides metathesizing a first alkenyl group with a first alkynyl group. In further embodiments, the present disclosure provides metathesizing a first alkynyl group and a second alkynyl group. In further embodiments, the present disclosure provides metathesizing a first alkenyl group, a second alkenyl group, and a third alkenyl group. In still other embodiments, the present disclosure provides metathesizing a first alkenyl group, a second alkenyl group, and a first alkynyl group. In yet further embodiments, the present disclosure provides metathesizing a first alkenyl group, a first alkynyl group, and a second alkynyl group.

The points of unsaturation may be present in the same molecule, thereby effecting a ring closing metathesis or intramolecular ring closing. As such, the single compound comprises at least the first alkenyl or alkynyl group and the second alkenyl or alkynyl group. The alkenyl and/or alkynyl group may be a terminal or internal group. The single compound may also contain other points of unsaturation or substituents that do not interfere with the metathesis.

In some embodiments, the one or more compound that can undergo a metathesis is of Formula (X1)-(X5):

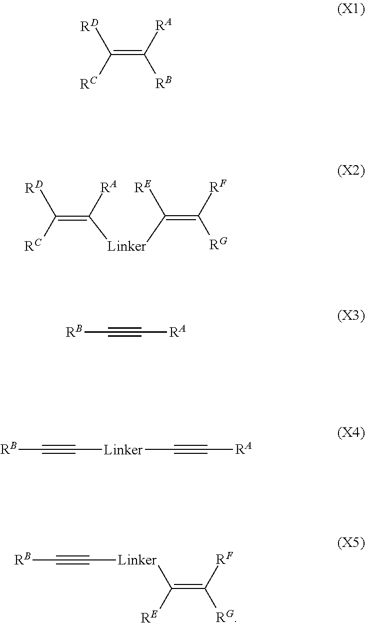

In these compounds, R^{A} to R^{G} are, independently, H, optionally substituted C₁₋₁₂alkyl, optionally substituted C₂₋₁₂alkenyl, optionally substituted C₂₋₁₂alkynyl, optionally substituted C₁₋₁₂haloalkyl, optionally substituted C₁₋₁₂heteroalkyl, optionally substituted C₃₋₁₂cycloalkyl, optionally substituted aryl, optionally substituted heteroaryl, optionally substituted heterocyclyl, OH, sulfonyl, CN, NO₂, halo, amino, C(O)H, COOH, acyl, carboxyl, amido, silyl, boryl, stannyl, or germyl. In other embodiments, R^{A} to R^{G} are, independently, H, halo (e.g., F, Cl, Br, I), OH, sulfhydryl, alkoxy, aryloxy, aralkyloxy, acyl (including alkylcarbonyl, arylcarbonyl, acyloxy (alkylcarbonyloxy and arylcarbonyloxy), alkoxycarbonyl, aryloxycarbonyl, halocarbonyl, carboxy, carboxylate), carbamoyl, mono-(alkyl)-substituted carbamoyl, di-(alkyl)-substituted carbamoyl, mono-(aryl)-substituted carbamoyl, di-(aryl)substituted, di-N-(alkyl),N-(aryl)-substituted carbamoyl, thiocarbamoyl, mono-(alkyl)-substituted thiocarbamoyl, di-(alkyl)-substituted thiocarbamoyl, mono-(aryl)substituted thiocarbamoyl, di-(aryl)-substituted thiocarbamoyl, di-N-(alkyl), N-(aryl)-substituted thiocarbamoyl, CN, OCN, thiocyanato, formyl, C(S)H, NH₂, mono-(alkyl)-substituted amino, di-(alkyl)-substituted amino, mono-(aryl)substituted amino, di-(aryl)-substituted amino, alkylamido, arylamido, NO₂, NO, alkylthio (—S-alkyl), arylthio (—S-aryl), alkyl, alkenyl, alkynyl, aryl, or aralkyl.

The Linker is absent or may be optionally substituted C₁₋₂₀alkyl, optionally substituted C₁₋₂₀heteroalkyl, optionally substituted C₃₋₁₂cycloalkyl, optionally substituted aryl, optionally substituted heteroaryl, optionally substituted heterocyclyl, sulfonyl, amino, carboxyl, or amido. In some embodiments, the Linker is a size that provides a product having about 4 to about 10 atoms. In other embodiments, the linker is a size that provides a product having about 5 to about 8 atoms, i.e., 5 atoms, 6 atoms, 7 atoms, or 8 atoms. Thus, in some embodiments, the linker has about 3 atoms, 4 atoms, 5 atoms, or 6 atoms.

In some embodiments, the compound is:

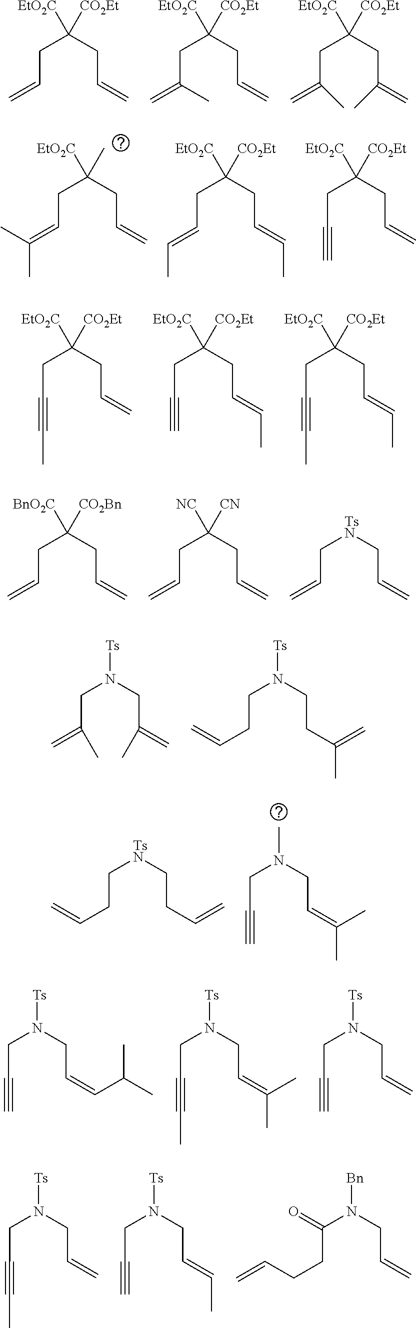

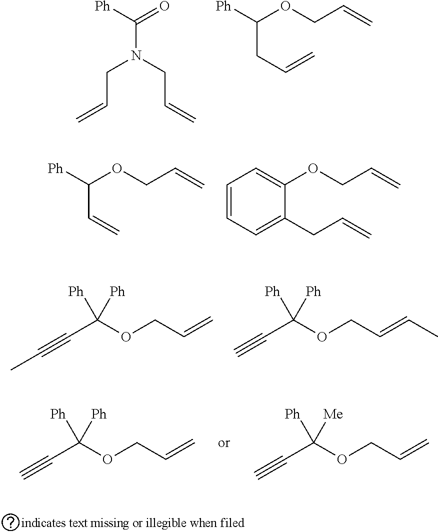

Alternatively, the points of unsaturation may be in two or more molecules. As such a first compound contains one point of unsaturation and a second compound contains the second point of unsaturation. Thus, the metathesis is an intermolecular reaction. In some embodiments, the first compound comprises the first alkenyl or alkynyl group and the second compound comprises the second alkenyl or alkynyl group. The alkenyl and/or alkynyl group may be a terminal or internal group. The single compound may also contain other points of unsaturation or substituents that do not interfere with the metathesis.

In some embodiments, the first compound and second compound are independently (X1)-(X5):

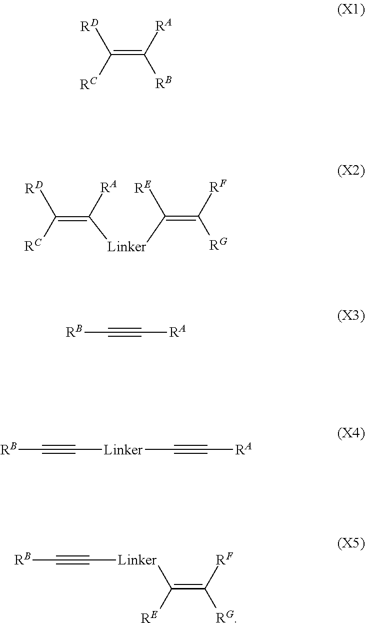

In these compounds, R^{A} to R^{G} are, independently, H, optionally substituted C₁₋₁₂alkyl, optionally substituted C₂₋₁₂alkenyl, optionally substituted C₂₋₁₂alkynyl, optionally substituted C₁₋₁₂haloalkyl, optionally substituted C₁₋₁₂heteroalkyl, optionally substituted C₃₋₁₂cycloalkyl, optionally substituted aryl, optionally substituted heteroaryl, optionally substituted heterocyclyl, OH, sulfonyl, CN, NO₂, halo, amino, C(O)H, COOH, acyl, carboxyl, amido, silyl, boryl, stannyl, or germyl. In other embodiments, R^{A} to R^{G} are, independently, H, halo (e.g., F, Cl, Br, I), OH, sulfhydryl, alkoxy, aryloxy, aralkyloxy, acyl (including alkylcarbonyl, arylcarbonyl, acyloxy (alkylcarbonyloxy and arylcarbonyloxy), alkoxycarbonyl, aryloxycarbonyl, halocarbonyl, carboxy, carboxylate), carbamoyl, mono-(alkyl)-substituted carbamoyl, di-(alkyl)-substituted carbamoyl, mono-(aryl)-substituted carbamoyl, di-(aryl)substituted, di-N-(alkyl),N-(aryl)-substituted carbamoyl, thiocarbamoyl, mono-(alkyl)-substituted thiocarbamoyl, di-(alkyl)-substituted thiocarbamoyl, mono-(aryl)substituted thiocarbamoyl, di-(aryl)-substituted thiocarbamoyl, di-N-(alkyl), N-(aryl)-substituted thiocarbamoyl, CN, OCN, thiocyanato, formyl, C(S)H, NH₂, mono-(alkyl)-substituted amino, di-(alkyl)-substituted amino, mono-(aryl) substituted amino, di-(aryl)-substituted amino, alkylamido, arylamido, NO₂, NO, alkylthio (—S— alkyl), arylthio (—S-aryl), alkyl, alkenyl, alkynyl, aryl, or aralkyl.

The Linker is absent or may be optionally substituted C₁₋₂₀alkyl, optionally substituted C₁₋₂₀heteroalkyl, optionally substituted C₃₋₁₂cycloalkyl, optionally substituted aryl, optionally substituted heteroaryl, optionally substituted heterocyclyl, sulfonyl, amino, carboxyl, or amido. In some embodiments, the Linker is a size that provides a product having about 4 to about 10 atoms. In other embodiments, the linker is a size that provides a product having about 5 to about 8 atoms, i.e., 5 atoms, 6 atoms, 7 atoms, or 8 atoms. Thus, in some embodiments, the linker has about 3 atoms, 4 atoms, 5 atoms, or 6 atoms.

In other embodiments, the two of more compound are selected from among:

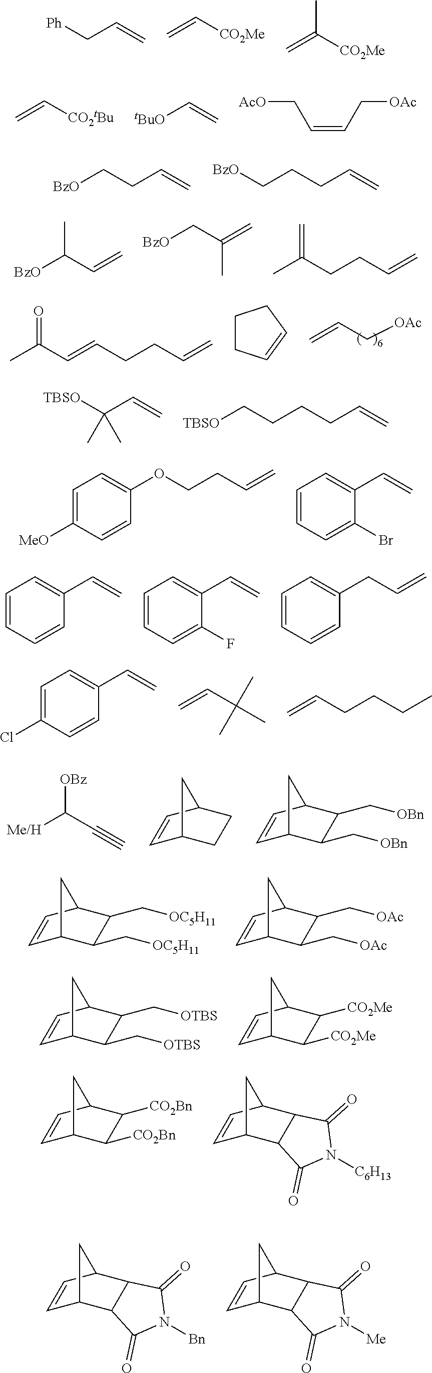

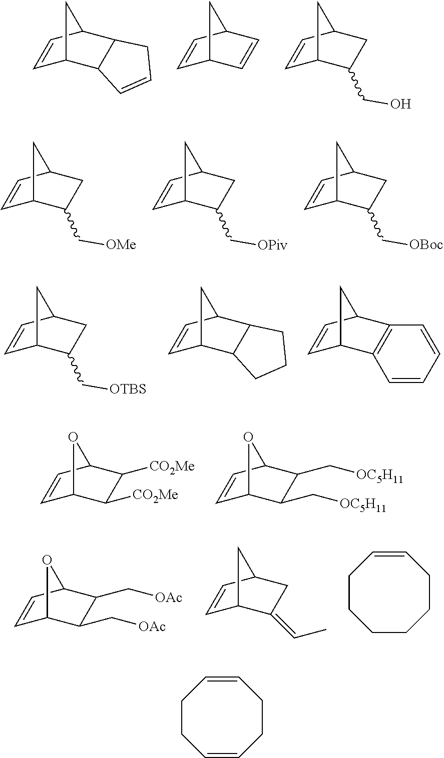

### Metathesis Methods

As described herein, the present disclosure is directed to methods for chemical metathesis that can be performed easily, with high yields, and at ambient temperatures. The methods include the use of infrared light (such as near infrared light), a ruthenium metathesis catalyst and a photo-redox catalyst that is activated by deep-red light or infrared light. In some embodiments, these methods include applying deep-red light or infrared light to one compound comprising a first alkenyl or alkynyl group and a second alkenyl or alkynyl group. In other embodiments, these methods include applying deep-red light or infrared light to a first compound comprising a first alkenyl or alkynyl group and a second compound comprising a second alkenyl or alkynyl group. The deep-red light or infrared light is applied to the compounds in the presence of the ruthenium metathesis catalyst and photo-redox catalyst.

The metathesis may be any type of chemical reaction that exchanges chemical bonds to result in one or more products that differ from the reactants. Thus, the metathesis may be a ring-closing metathesis, cross-metathesis, ring-opening metathesis polymerization, photolithographic olefin metathesis polymerization. In some embodiments, the methods described herein relate to ring-closing metathesis, i.e., an intramolecular metathesis. In other embodiments, the methods described herein relate to cross-metathesis, i.e., an intermolecular metathesis. In further embodiments, the methods described herein related to ring-opening metathesis polymerization. In yet other embodiments, the methods relate to photolithographic olefin metathesis polymerization.

Advantageously, the methods discussed herein may be performed at a range of temperatures without an adverse effect of the yield or conversion. In some embodiments, the metathesis is performed at a temperature of about −80 to about 200° C. In further embodiments, the metathesis may be performed at about −80° C., about −75° C., about −70° C., about −65° C., about −60° C., about −55° C., about −50° C., about −45° C., about −40° C., about −35° C., about −30° C., about −25° C., about −20° C., about −15° C., about −10° C., about −5° C., about 0° C., about 10° C., about 15° C., 20° C., about 25° C., about 30° C., about 35° C., about 40° C., about 45° C., about 50° C., about 55° C., about 60° C., about 65° C., about 70° C., about 75° C., about 80° C., about 85° C., about 90° C., about 95° C., about 100° C., about 105° C., about 110° C., about 115° C., about 120° C., about 125° C., about 130° C., about 135° C., 140° C., about 145° C., about 150° C., about 155° C., about 160° C., about 165° C., about 170° C., about 175° C., about 180° C., about 185° C., about 190° C., about 195° C., or about 200° C. In other embodiments, the metathesis may be performed at a temperature at about −80 to about 180° C., about −80 to about 150° C., about −80 to about 125° C., about −80 to about 100° C., about −80 to about 75° C., about −80 to about 50° C., about −80 to about 25° C., about −80 to about 0° C., about −80 to about −20° C., about −30 to about 180° C., about −30 to about 150° C., about −30 to about 125° C., about −30 to about 100° C., about −30 to about 75° C., about −30 to about 50° C., about −30 to about 25° C., about −30 to about 0° C., about −30 to about −20° C., about −10 to about 180° C., about −10 to about 150° C., about −10 to about 125° C., about −10 to about 100° C., about −10 to about 75° C., about −10 to about 50° C., about −10 to about 25° C., about −10 to about 0° C., about 0 to about 180° C., about 0 to about 150° C., about 0 to about 125° C., about 0 to about 100° C., about 0 to about 75° C., about 0 to about 50° C., about 0 to about 25° C., 10 to about 180° C., about 10 to about 150° C., about 10 to about 125° C., about 10 to about 100° C., about 10 to about 75° C., about 10 to about 50° C., or about 10 to about 25° C. In yet other embodiments, the metathesis is performed at room temperature. In still further embodiments, the metathesis is performed at a temperature of about 20 to about 30° C.

The amount of the ruthenium metathesis catalyst and/or photo-redox catalyst depends on the compound to be metathesized and product to be prepared. In some embodiments, lower amounts of the ruthenium metathesis catalyst and/or photo-redox catalyst are used. In other embodiments, it is contemplated that higher amounts of the ruthenium metathesis catalyst and/or photo-redox catalyst may be required. Thus, in some embodiments, the metathesis is performed using about 0.01 to about 10 mol %, based on the mol % of the one compound or first and second compound, of the ruthenium metathesis catalyst. In other embodiments, the metathesis is performed using about 0.05 to about 10 mol %, about 1 to about 10 mol %, about 1.5 to about 10 mol %, about 2 to about 10 mol %, about 2.5 to about 10 mol %, about 3 to about 10 mol %, about 3.5 to about 10 mol %, about 4 to about 10 mol %, about 4.5 to about 10 mol %, about 5 to about 10 mol %, about 5.5 to about 10 mol %, about 6 to about 10 mol %, about 6.5 to about 10 mol %, about 7 to about 10 mol %, about 7.5 to about 10 mol %, about 8 to about 10 mol %, about 8.5 to about 10 mol %, about 9 to about 10 mol %, about 0.01 to about 9 mol %, about 0.01 to about 8 mol %, about 0.01 to about 7 mol %, about 0.01 to about 6 mol %, about 0.01 to about 5 mol %, about 0.01 to about 4 mol %, about 0.01 to about 3 mol %, about 0.01 to about 2 mol %, about 0.01 to about 1 mol %, about 2 to about 7.5 mol %, about 2.5 to about 7.5 mol %, about 5 to about 7 mol %, or about 5 to about 10 mol %, based on the mol % of the one compound or first and second compound, of the ruthenium metathesis catalyst. In further embodiments, the metathesis is performed using about 2 to about 7.5 mol %, based on the mol % of the one compound or first and second compound, of the ruthenium metathesis catalyst. In still other embodiments, the metathesis is performed using about 5 mol %, based on the mol % of the one compound or first and second compound, of the ruthenium metathesis catalyst.

Thus, in some embodiments, the metathesis is performed using about 0.05 to about 10 mol %, based on the mol % weight of the one compound or first and second compound, of the photo-redox catalyst. In other embodiments, the metathesis is performed using about 1 to about 10 mol %, about 1.5 to about 10 mol %, about 2 to about 10 mol %, about 2.5 to about 10 mol %, about 3 to about 10 mol %, about 3.5 to about 10 mol %, about 4 to about 10 mol %, about 4.5 to about 10 mol %, about 5 to about 10 mol %, about 5.5 to about 10 mol %, about 6 to about 10 mol %, about 6.5 to about 10 mol %, about 7 to about 10 mol %, about 7.5 to about 10 mol %, about 8 to about 10 mol %, about 8.5 to about 10 mol %, about 9 to about 10 mol %, about 0.05 to about 9 mol %, about 0.05 to about 8 mol %, about 0.05 to about 7 mol %, about 0.05 to about 6 mol %, about 0.05 to about 5 mol %, about 0.05 to about 4 mol %, about 0.05 to about 3 mol %, about 0.05 to about 2 mol %, about 0.05 to about 1 mol %, about 2 to about 7.5 mol %, about 2.5 to about 7.5 mol %, about 5 to about 7 mol %, or about 5 to about 10 mol %, based on the mol % of the one compound or first and second compound, of the photo-redox catalyst. In further embodiments, the metathesis is performed using about 2 to about 7.5 mol %, based on the mol % of the one compound or first and second compound, of the photo-redox catalyst. In still other embodiments, the metathesis is performed using about 7.5 mol %, based on the mol % of the one compound or first and second compound, of the photo-redox catalyst.

The inventors also found that the concentration of the one or more compounds containing the points of unsaturation can be adjusted to optimize the metathesis. In some embodiments, the concentration of the one compound or first and second compound, all containing points of unsaturation, is about 0.01 to about 5M. In other embodiments, the concentration is about 0.01 to about 4.5M, about 0.01 to about 4M, about 0.01 to about 3.5M, about 0.01 to about 3M, about 0.01 to about 2.5M, about 0.01 to about 2M, about 0.01 to about 1.5M, about 0.01 to about 1M, about 0.01 to about 0.05M, about 0.5M to about 5M, about 0.1 to about 1M, about 0.1 to about 0.8M, about 0.1 to about 0.75M, about 0.1 to about 0.5M, about 0.1 to about 0.4M, about 0.1 to about 0.3M, about o, 1 to about 0.2M, about 1 to about 5M, about 1.5 to about 5M, about 2 to about 5M, about 2.5 to about 5M, about 3 to about 5M, about 3.5 to about 5M, about 4 to about 5M, or about 4.5 to about 5M. In further embodiments, the concentration is about 0.01 to about 0.5 M. In yet other embodiments, the concentration is about 0.1 to about 0.3 M.

The metathesis is performed for a period of time as determined by those skilled in the art depending on the ruthenium catalyst, photo-redox catalyst, temperature, and one or more compounds to be metathesized. The reaction time can be varied as needed to control the thickness of the metathesized compound, among others. Thus, shorter periods of times may result in thinner polymers, whereas longer periods of time may result in thicker polymers. In some embodiments, the metathesis is performed for at least about 10 seconds. In other embodiments, the metathesis is performed for at least about 1 minute. In further embodiments, the metathesis is performed for at least about 2 minutes, 3 minutes, 4 minutes, 5 minutes, 10 minutes, 30 minutes, 60 minutes, 2 hours, 3 hours, 4 hours, 5 hours, 6 hours, 12 hours, or 24 hours, or longer.

### Spatial Control Methods

In addition to the fact that the metatheses described herein may be performed with high yields and conversions, optionally at ambient conditions when needed, the inventors found that they could be spatially controlled. As such, methods for spatially controlling a metathesis also are provided by the disclosure, as are methods for preparing polymeric materials or patterns, and patterning surfaces on a micro scale as described below.

The term “micro scale” as used herein refers to patterned polymers having a size of about 1 mm or less. The size may be in any direction of the pattern, i.e., width, length or depth. In some embodiments, micro scale refers to a size of about 1 nm to about 1 mm. In other embodiments, micro scale refers to a size of about 1 nm to about 1 μm. In further embodiments, micro scale refers to a size of about 1 μm to about 1 mm. In still other embodiments, micro scale refers to a size of about 10 to about 100 μm, about 20 to about 100 μm, about 30 to about 100 μm, about 40 to about 100 μm, about 50 to about 100 μm, about 60 to about 100 μm, about 70 to about 100 μm, about 80 to about 100 μm, about 90 to about 100 μm, about 10 to about 90 μm, about 10 to about 80 μm, about 10 to about 80 μm, about 10 to about 70 μm, about 10 to about 60 μm, about 10 to about 50 μm, about 10 to about 40 μm, about 10 to about 30 μm, about 10 to about 20 μm, about 20 to about 90 μm, about 20 to about 80 μm, about 20 to about 70 μm, about 20 to about 60 μm, about 20 to about 50 μm, about 20 to about 40 μm, about 20 to about 30 μm, about 30 to about 90 μm, about 30 to about 80 μm, about 30 to about 70 μm, about 30 to about 60 μm, about 30 to about 60 μm, about 30 to about 50 μm, about 30 to about 40 μm, about 40 to about 90 μm, about 30 to about 80 μm, about 30 to about 70 μm, about 30 to about 60 μm, about 30 to about 50 μm, about 30 to about 40 μm, about 40 to about 90 μm, about 40 to about 80 μm, about 40 to about 70 μm, about 40 to about 60 μm, about 40 to about 50 μm, about 50 to about 90 μm, about 50 to about 80 μm, about 50 to about 70 μm, about 50 to about 60 μm, about 60 to about 90 μm, about 60 to about 80 μm, about 60 to about 70 μm, about 70 to about 90 μm, about 70 to about 80 μm, or about 80 to about 90 μm.

Methods of spatially controlling a metathesis comprise forming a mixture of a ruthenium metathesis catalyst, a photo-redox catalyst, and one or more compounds susceptible to metathesis and applying infrared light to one or more regions of the mixture. By doing so, the methods provide one or more metathesized regions and one or more un-metathesized regions.

The mixture may be formed or added to a substrate that contains the mixture. In some embodiments, the substrate is a glass, plastic, an organic surface including a metal such as gold, or iron, cloth, wood, silicon, diamond, graphite, charcoal, metal organic framework, among others. In some embodiments, the substrate is a petri dish. In other embodiments, the substrate is a wafer, such as a silicon wafer. As such, the substrate does not participate in the metathesis or otherwise form any bonds with the one or more compounds, ruthenium metathesis catalyst, photo-redox catalyst, or product formed therefrom.

Thus, in certain embodiments, the mixture may be disposed on the substrate prior to metathesis. In further embodiments, the mixture is disposed on a substrate that does not participate in the metathesis.

The disclosure also envisions embodiments wherein substrate is functionalized with the one or more compounds susceptible to metathesis, ruthenium metathesis catalyst, photo-redox catalyst, or combinations thereof. By doing so, the substrate is linked to a metathesized region. In some embodiments, the substrate is functionalized with the one or more compounds susceptible to metathesis. In other embodiments, the substrate is pre-functionalized with the one or more compound before adding the photo-redox catalyst or ruthenium metathesis catalyst. In further embodiments, the substrate is functionalized with the photo-redox catalyst. In yet other embodiments, the substrate is functionalized with the ruthenium metathesis catalyst. As but one example, the present disclosure provides methods that comprise applying deep-red light or infrared light to a ruthenium metathesis catalyst, a photo-redox catalyst, and one or more compounds susceptible to metathesis, the applying being performed so as to give rise to one or more metathesized regions, at least one of the ruthenium metathesis catalyst and the photo-redox catalyst (or even both of the foregoing) being linked to a substrate, the substrate optionally being stationary. The deep-red light or infrared light can be applied in a predetermined pattern. The deep-red light or infrared light can also be applied from two or more sources.

The deep-red light or infrared light may be applied using any light source known in the art. On some embodiments, the deep-red light or infrared light is applied using a high resolution light source. The term “high resolution” as used herein refers to light that is delivered to a specific location on a substrate. In some embodiments, the high resolution light source is a laser. In other embodiments, the high resolution light source is a fine beam of light.

In order to spatially control the metathesis, the regions that are not to be metathesized, i.e., the un-metathesized regions, are covered with a photomask. By doing so, the light penetrates the mask in intended locations as determined by the shape and placement of the mask. The terms “mask” and “photomask” as used herein are interchangeable and refer to an object that physically covers regions of the compounds susceptible to metathesis. Desirably, the mask is substantially opaque to infrared light. In some embodiments, the mask is black in color or made of a material that reflects infrared light. In other embodiments, the mask is black paper, black plastic, a metal sheet, or a metal foil. The mask may also have one or more openings whereby infrared light may pass through. By doing so, those openings permit the infrared light to only be applied to those regions of the mixture that are intended to metathesize. As the final product, the mixture will contain un-metathesized and metathesized regions.

The disclosure also provides steps for recovering only those metathesized regions. In doing so, the un-metathesized regions may be removed by rinsing with a solvent. In some embodiments, the solvent is added to the mixture and thereby removed to provide the metathesized region. The solvent selected desirably is effect to only solubilize the un-metathesized regions, and not the metathesized regions. One skilled in the art would understand how long the rinsing should be performed and how best to remove the solvent after rinsing.

A further embodiment of the present disclosure is a method of exerting spatial control over metathesis comprising: (a) providing a reaction mixture of a latent metathesis catalyst, a photocatalyst, and a substrate; and (b) applying deep-red light or infrared light to selected areas of the reaction mixture; wherein the selected areas of the reaction mixture are selected by: (i) macroscopic or microscopic photomask; or (ii) high resolution light source. In some embodiments, the reaction mixture is provided on a support surface. In certain embodiments, the support surface is a pre-functionalized support surface.

Another embodiment of the present disclosure is a method for deep-red light or infrared light controlled olefin metathesis, comprising: (a) providing a reaction mixture of a latent metathesis catalyst, a photocatalyst, and a substrate; and (b) applying deep-red light or infrared light to the reaction mixture for a desired time. In some embodiments, the olefin metathesis is selected from ring-closing metathesis (RCM), cross-metathesis (CM), and ring-opening metathesis polymerization (ROMP). In some embodiments, step (b) of the method disclosed above is carried out at room temperature.

The present disclosure further provides compositions and processes as disclosed or depicted in the Appendix attached hereto, and/or kits containing such compositions or for carrying out such processes.

The embodiments described in this disclosure can be combined in various ways. Any aspect or feature that is described for one embodiment can be incorporated into any other embodiment mentioned in this disclosure. While various novel features of the inventive principles have been shown, described and pointed out as applied to particular embodiments thereof, it should be understood that various omissions and substitutions and changes may be made by those skilled in the art without departing from the spirit of this disclosure. Those skilled in the art will appreciate that the inventive principles can be practiced in other than the described embodiments, which are presented for purposes of illustration and not limitation.

The following listing of aspects is intended to complement, rather than displace or supersede, the previous descriptions.

### Aspects

Aspect 1: A composition for metathesizing a first alkenyl or alkynyl group with a second alkenyl or alkynyl group, the composition comprising: a ruthenium metathesis catalyst and a photo-redox catalyst that is activated by deep-red light or infrared light.

Aspect 2: The composition of aspect 1, wherein the deep-red light has a wavelength of from about 600 nm to about 720 nm and the infrared light has a wavelength of about 720 nm to about 900 nm.

Aspect 3: The composition of aspect 1, wherein the ruthenium catalyst is of Formula (I) or Formula (II):

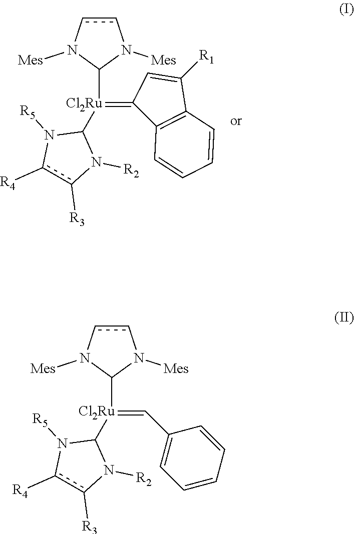

wherein:

R¹ is H, C₁₋₆alkyl, C₂₋₆alkenyl, aryl, or heteroaryl;

R² to R⁵ are, independently, H, C₁₋₆alkyl, C₁₋₆alkoxy, halo, or aryl; and

Mes is

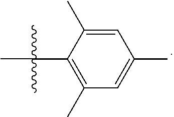 [SMILES: *c1c(C)cc(C)cc1C]

Aspect 4: The composition of aspect 1, wherein the ruthenium catalyst is

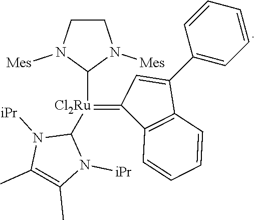

Aspect 5: The composition of aspect 1, wherein the ruthenium catalyst is

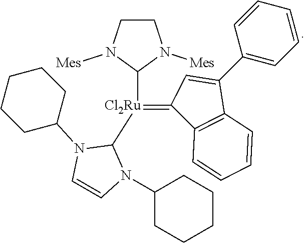

Aspect 6: The composition of aspect 1, wherein the ruthenium catalyst is

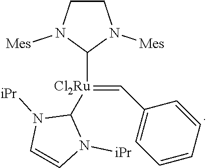

Aspect 7: The composition of aspect 1, wherein the photo-redox catalyst is highly oxidizing.

Aspect 8: The composition of aspect 1, wherein the photo-redox catalyst comprises Osmium.

Aspect 9: The composition of aspect 1, wherein the photo-redox catalyst is Os(phen)₃(PF₆)₂; wherein phen is 1,10 phenanthroline.

Aspect 10: The composition of aspect 1, wherein the photo-redox catalyst is selected from:

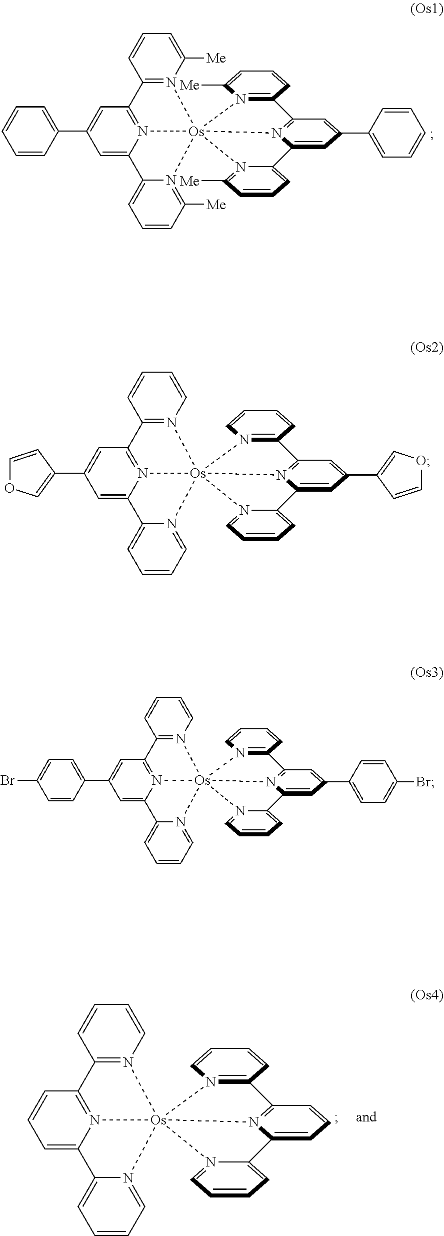

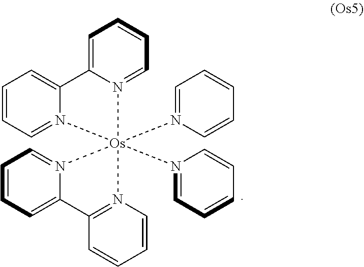

Aspect 11: A method for chemical metathesis, comprising applying deep-red light or infrared light to (i) one compound comprising a first alkenyl or alkynyl group and a second alkenyl or alkynyl group or (ii) a first compound comprising a first alkenyl or alkynyl group and a second compound comprising a second alkenyl or alkynyl group, in the presence of a ruthenium metathesis catalyst and a photo-redox catalyst that is activated by deep-red light or infrared light.

Aspect 12: The method of aspect 11, wherein the deep-red light has a wavelength of from about 600 nm to about 720 nm and the infrared light has a wavelength of about 720 nm to about 900 nm.

Aspect 13: The method of aspect 11, wherein the ruthenium catalyst is of Formula (I) or Formula (II):

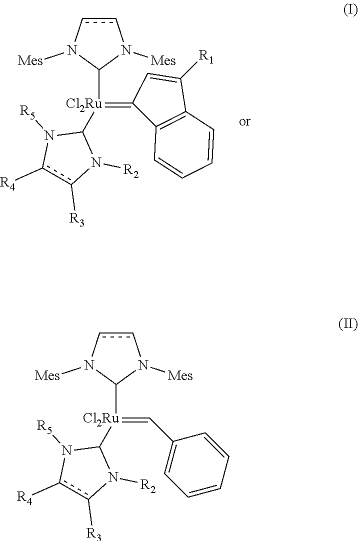

wherein:

R¹ is H, C₁₋₆alkyl, C₂₋₆alkenyl, aryl, or heteroaryl;

R² to R⁵ are, independently, H, C₁₋₆alkyl, C₁₋₆alkoxy, halo, or aryl; and

Mes is

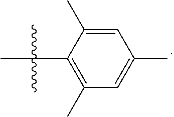 [SMILES: *c1c(C)cc(C)cc1C]

Aspect 14: The method of aspect 11, wherein the ruthenium catalyst is

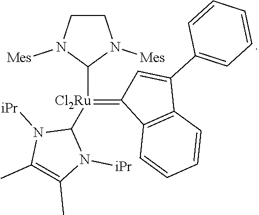

Aspect 15: The method of aspect 11, wherein the ruthenium catalyst is

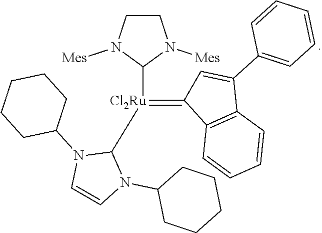

Aspect 16: The method of aspect 11, wherein the ruthenium catalyst is

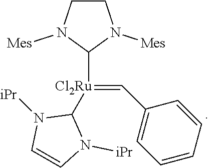

Aspect 17: The method of aspect 11, wherein the photo-redox catalyst is highly oxidizing.

Aspect 18: The method of aspect 11, wherein the photo-redox catalyst comprises Osmium.

Aspect 19: The method of aspect 11, wherein the photo-redox catalyst is Os(phen)₃(PF₆)₂; wherein phen is 1,10 phenanthroline.

Aspect 20: The method of aspect 11, wherein the photo-redox catalyst is selected from:

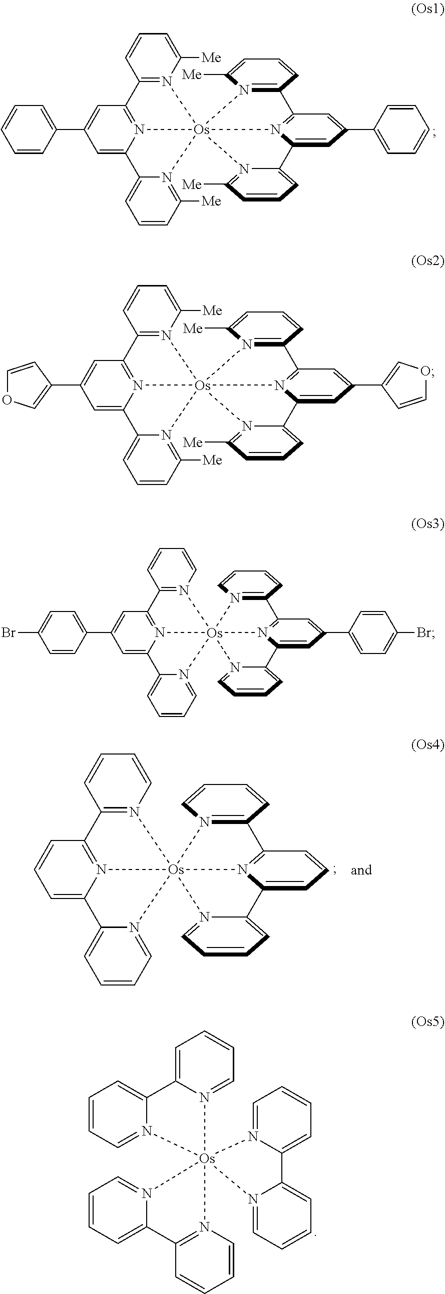

Aspect 21: The method of aspect 11, that is performed at a temperature of about 20 to about 30° C.

Aspect 22: The method of aspect 11, comprising about 0.01 to about 10 mol %, based on the mol % of the one compound or first and second compound, of the ruthenium metathesis catalyst.

Aspect 23: The method of aspect 11, wherein the concentration of the one compound or first and second compound is about 0.01 to about 5 M.

Aspect 24: A method of spatially controlling a metathesis, comprising:

forming a mixture of a ruthenium metathesis catalyst, a photo-redox catalyst, and one or more compounds susceptible to metathesis; and applying deep-red light or infrared light to one or more regions of the mixture so as to give rise to one or more metathesized regions and one or more un-metathesized regions.

Aspect 25: The method of aspect 24, wherein the deep-red light or infrared light is applied using a high resolution light source.

Aspect 26: The method of aspect 24, wherein at least one of the un-metathesized regions is covered with a photomask.

Aspect 27: The method of aspect 24, wherein the mixture is disposed on a substrate that is functionalized with the one or more compounds susceptible to metathesis.

Aspect 28: A method of spatially controlling a metathesis, comprising:

applying deep-red light or infrared light to a ruthenium metathesis catalyst, a photo-redox catalyst, and one or more compounds susceptible to metathesis, the applying being performed so as to give rise to one or more metathesized regions, at least one of the ruthenium metathesis catalyst and the photo-redox catalyst being linked to a substrate, the substrate optionally being stationary.

Aspect 29: The method of aspect 28, wherein the deep-red light or infrared light is applied in a predetermined pattern.

The following Examples are provided to illustrate some of the concepts described within this disclosure. While each Example is considered to provide specific individual embodiments of composition, methods of preparation and use, none of the Examples should be considered to limit the more general embodiments described herein.

In the following examples, efforts have been made to ensure accuracy with respect to numbers used (e.g. amounts, temperature, etc.) but some experimental error and deviation should be accounted for. Unless indicated otherwise, temperature is in degrees C., pressure is at or near atmospheric.

### EXAMPLES

### Example 1

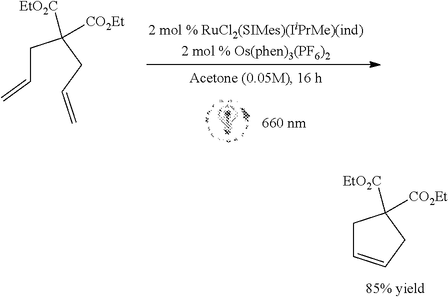 [SMILES: C=CCC(CC=C)(C(=O)OCC)C(=O)OCC ; CCOC(=O)C1(C(=O)OCC)CC=CC1]

Reaction of diethyl 2,2-diallylmalonate with 2 mol % of (SIMes)(IiPrMe)(ind)RuCl₂ and 2 mol % Os(phen)₃(PF₆)₂ in acetone under deep-red light for 16 hours produced diethyl cyclopent-3-ene-1,1-dicarboxylate in 85% yield.

### Examples 2-6

The compounds shown below in Table 1 were subject to ring closing metathesis from their corresponding linear compounds according to the conditions described in Example 1.

TABLE 1
RCM and Yields

| Example | Compound | Yield |
|---|---|---|
| 2 | 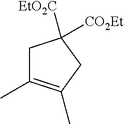 [SMILES: CCOC(=O)C1(C(=O)OCC)CC(C)=C(C)C1] | NR |
| 3 | 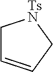 [SMILES: Cc1ccc(S(=O)(=O)N2CC=CC2)cc1] | 85% |
| 4 | 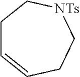 [SMILES: Cc1ccc(S(=O)(=O)N2CCC=CCC2)cc1] | 84% |
| 5 | 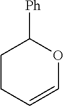 [SMILES: C1=COC(c2ccccc2)CC1] | 37% |
| 6 | 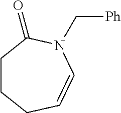 [SMILES: O=C1CCCC=CN1Cc1ccccc1] | 66% |

### Examples 7-8

The compounds shown below in Table 2 were subject to cross metathesis from their corresponding compounds according to the conditions described in Example 1.

TABLE 2
Cross-Metathesis and Yields

| Example | Compound | Yield |
|---|---|---|
| 7 | 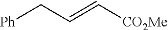 [SMILES: COC(=O)/C=C/Cc1ccccc1] | 30% |
| 8 | 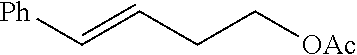 [SMILES: CC(=O)OCC/C=C/c1ccccc1] | 29% |

### Examples 9-10

The compounds shown below in Table 3 were subject to ring opening metathesis according to the conditions described in Example 1.

TABLE 3
ROMP and Yields

| Example | Compound | Yield |
|---|---|---|
| 9 | 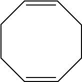 | >95% |
| 10 | 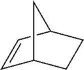 | >95% |

### Example 11

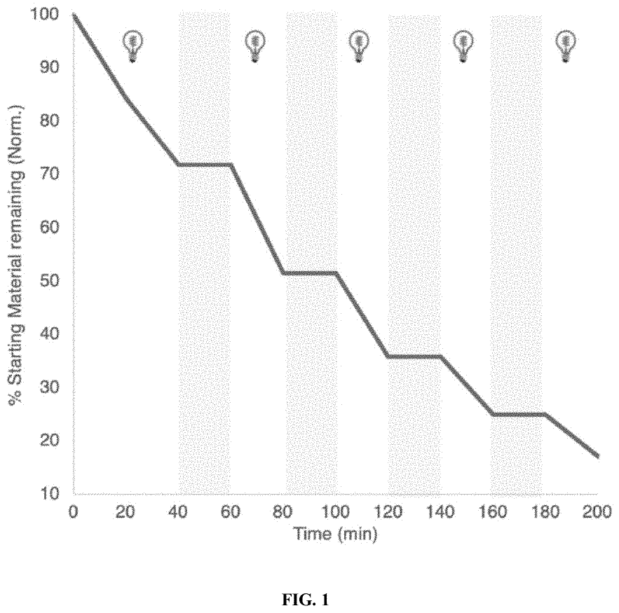

Temporal control of ROMP of cyclooctadiene through an in-situ LED-NMR on/off was observed for the reaction below. The reactivity is dependent on and controlled by light irradiation, as denoted by the lack of starting material consumption in the dark regions in FIG. 1.

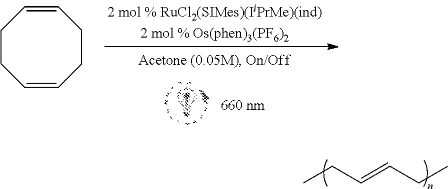

### Example 12

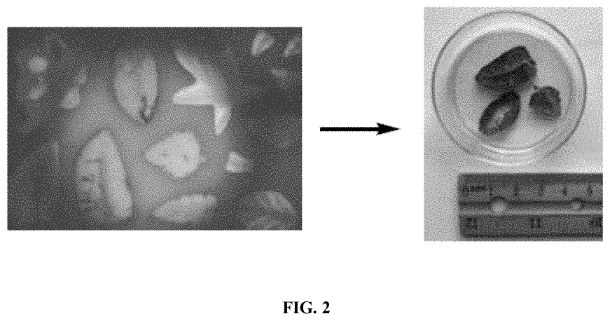

Spatial control of ROMP was observed for the reaction shown below and produces polydicyclopentadiene (pDCPD) in the shape of the mold, as shown in FIG. 2. Irradiation through a 7 mm silicone mold with 660 nm light formed leaf-like polymeric shapes and gelation was observed within about an hour.

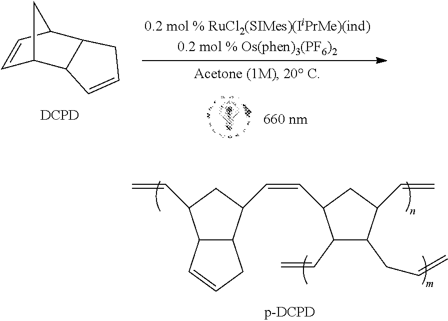

### Example 13

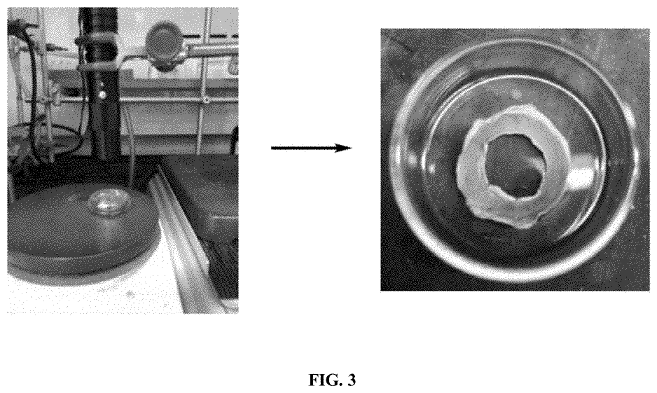

Spatial control of ROMP was observed for the reaction of Example 12 and produces polydicyclopentadiene (pDCPD) in the shape of the mold, as shown in FIG. 3. Irradiation with a red laser clamped to an orbital shaker formed circular polymeric shapes.

### Example 14

Spatial control of ROMP was observed for the reaction of Example 12. The polymerization and crosslinking of DCPD through several barrier penetration tests produces polydicyclopentadiene (pDCPD). Through amber glass, white paper, and a solution of Hemoglobin, free-standing gels of pDCPD were observed when using NIR light, but not with blue light. The results are summarized below in Table 4.

TABLE 4
Barrier Penetration Results

| Barrier | Red light (660 nm) | Blue light (456 nm) |
|---|---|---|
| Air | Gel | Gel |
| Amber Glass | Gel | No Gel |
| White Paper | Gel | No Gel |
| Hemoglobin (0.2 mM) | Gel | No Gel |

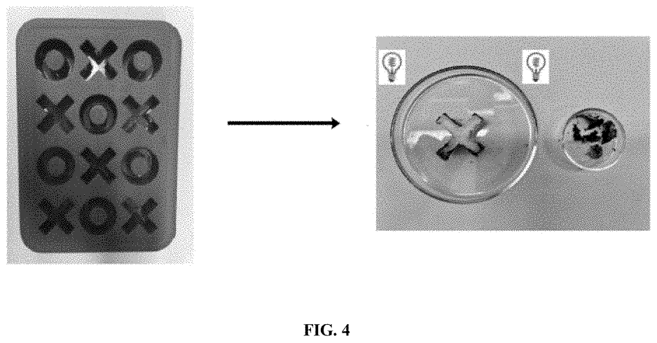

A larger scale polymerization was achieved by shining light onto a mold covered with a silicone pad, as shown in FIG. 4. The resulting polymers were shaped by the mold itself, indicating that the light had penetrated through the pad and initiated polymerization. Polymerization through the mold was attempted using blue light, which resulted in a thin film that could not be removed from the mold without complete destruction of its shape, while NIR light resulted in a 1 cm thick polymer.

### Examples 15-19

Reaction of diethyl 2,2-diallylmalonate was carried out as in Example 1, with 2 mol % of (SIMes)(IiPrMe)(ind)RuCl₂ and 2 mol % of osmium catalysts Os1, Os2, Os3, Os4 and Os5 in acetone under deep-red light for 16 hours produced diethyl cyclopent-3-ene-1,1-dicarboxylate in various yields, as shown in Table 5 below.

TABLE 5
RCM and Yields

| Example | Osmium Catalyst | Yield |
|---|---|---|
| 15 | 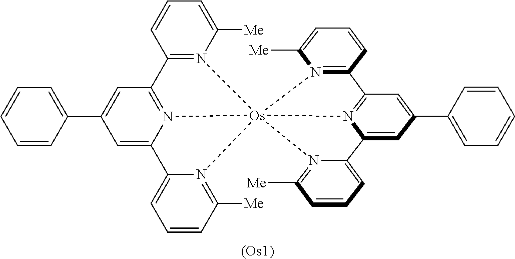 | 2% |
| 16 | 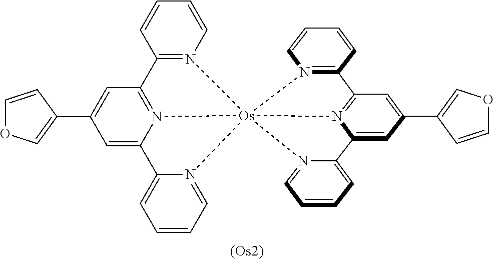 | 4% |
| 17 | 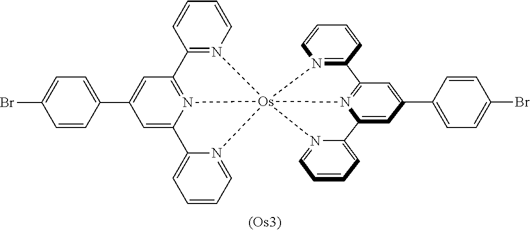 | 11% |
| 18 | 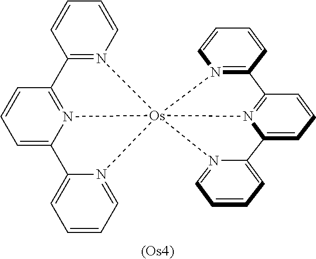 | 63% |
| 19 | 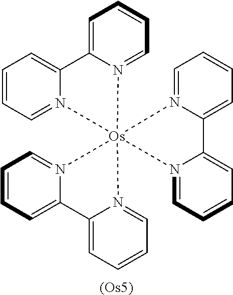 | 66% |

Similar yields for the olefin metathesis processes demonstrated in the Examples were obtained with light having a wavelength of 740 nm. The compounds and methods described herein demonstrate utility in the synthesis of robust polymers such as poly-dicyclopentadiene. When coupled with light, high levels of spatio-temporal control over polymerization can allow for formation of shapes with high resolution. Furthermore, with the flick of a switch, reactivity can be turned on and off.

### Example 20

Different catalyst loadings were studied in order to elucidate the nature of the On/Off behavior observed in the system shown below. The results are summarized in Table 6 below.

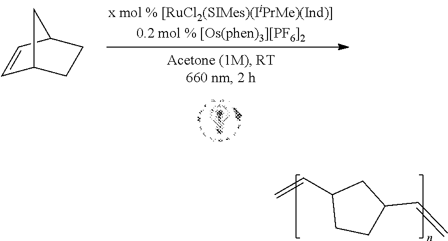

TABLE 6
Catalyst Loadings, Molecular Weights and PolyDispersity

| Entry | Ru Loading (mol %) | Mₙ (kDa)* | M_{w} (kDa)* | D |
|---|---|---|---|---|
| 1 | 0.2 | 411 | 660 | 1.61 |
| 2 | 0.02 | 573 | 876 | 1.53 |
| 3 | 0.002 | 1216 | 1354 | 1.11 |

*GPC data in THE with polystyrene standards

It is believed that there are two pathways through which this can proceed: 1) the reaction is deactivated during the Off periods; or 2) only a minute amount of the catalyst is activated and performs metathesis until it reaches its turnover number. In the latter case, new initiators can only be activated when the light is on. To test this hypothesis, the molecular weights (MW) of norbornene polymers were compared when polymerized at different loadings. From this GPC data, it appears that at different Ru concentrations, high MW polymers are still obtained, and it is most likely that only small amounts of catalyst are activated throughout the On/Off periods.

### Example 21

On/Off studies with norbornene were conducted to ensure that the initiator is saturated in the starting material in the system shown below. The second On period is longer to determine if a greater effect on the polydispersity index (B) can be realized. The results are summarized in Table 7 below.

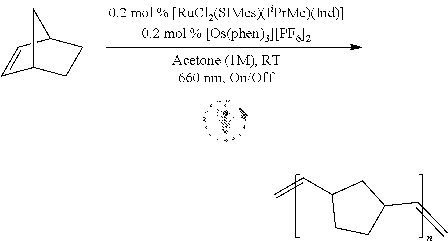

TABLE 7
On/Off Conditions, Polymer Growth and Polydispersity

| Entry | On/Off (min) | Mₙ (kDa)* | M_{w} (kDa)* | Ð |
|---|---|---|---|---|
| 1 | 30 On | 486 | 817 | 1.68 |
| 2 | 30 On, 30 Off | 738 | 919 | 1.24 |
| 3 | 30 On, 30 Off, 30 On | 393 | 783 | 1.99 |

*GPC data in THE with polystyrene standards

Ð is broad upon irradiation, indicating that initiators are slowly but steadily activated. However, with the light off and the catalyst saturated in monomer, it was observed that the length increases and Ð decreases. This indicates that the activated initiators are eventually reaching their turnover number. With this, it is believed that a slow initiation pathway is proceeding; however, only upon irradiation is reactivity restored to make more polymer of lengths comparable to the first irradiation period.

What is claimed:

1. A composition for metathesizing a first alkenyl or alkynyl group with a second alkenyl or alkynyl group, the composition comprising: a ruthenium metathesis catalyst and a photo-redox catalyst that is activated by deep-red light or infrared light.

2. The composition of claim 1, wherein the deep-red light has a wavelength of from about 600 nm to about 720 nm and the infrared light has a wavelength of about 720 nm to about 900 nm.

3. The composition of claim 1, wherein the ruthenium catalyst is of Formula (I) or Formula (II): 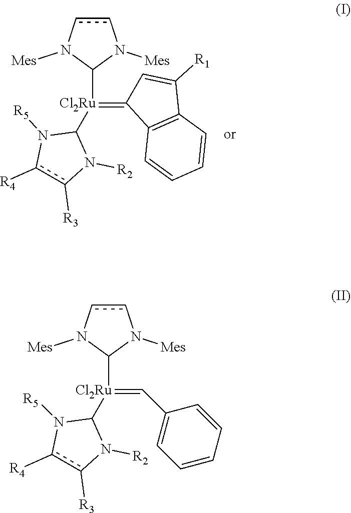
wherein:
R¹ is H, C₁₋₆alkyl, C₂₋₆alkenyl, aryl, or heteroaryl;
R² to R⁵ are, independently, H, C₁₋₆alkyl, C₁₋₆alkoxy, halo, or aryl; and
Mes is

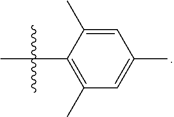 [SMILES: *c1c(C)cc(C)cc1C]

4. The composition of claim 1, wherein the ruthenium catalyst is

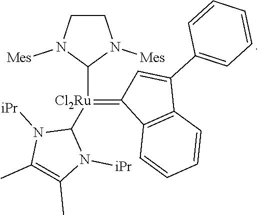

5. The method of claim 1, wherein the ruthenium catalyst is

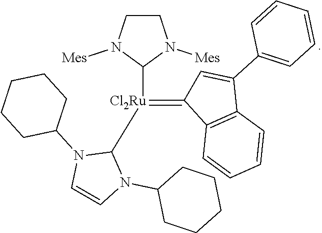

6. The method of claim 1, wherein the ruthenium catalyst is

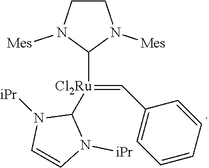

7. The composition of claim 1, wherein the photo-redox catalyst is highly oxidizing.

8. The composition of claim 1, wherein the photo-redox catalyst comprises osmium.

9. The composition of claim 1, wherein the photo-redox catalyst is Os(phen)₃(PF₆)₂; wherein phen is 1,10 phenanthroline.

10. The composition of claim 1, wherein the photo-redox catalyst is selected from:

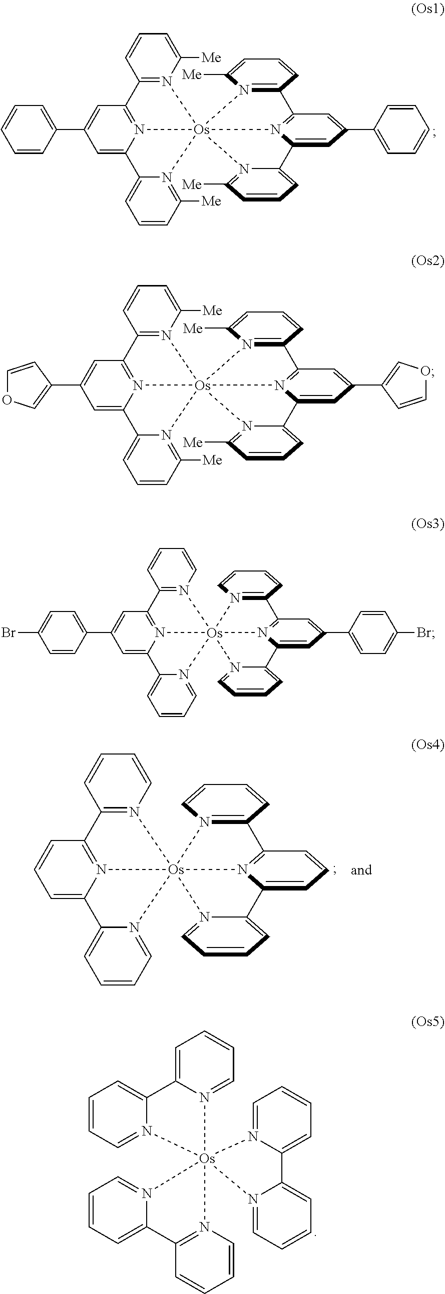

11. A method for chemical metathesis, comprising applying deep-red light or infrared light to (i) one compound comprising a first alkenyl or alkynyl group and a second alkenyl or alkynyl group or (ii) a first compound comprising a first alkenyl or alkynyl group and a second compound comprising a second alkenyl or alkynyl group, in the presence of a ruthenium metathesis catalyst and a photo-redox catalyst that is activated by deep-red light or infrared light.

12. The method of claim 11, wherein the deep-red light has a wavelength of from about 600 nm to about 720 nm and the infrared light has a wavelength of about 720 nm to about 900 nm.

13. The method of claim 11, wherein the ruthenium catalyst is of Formula (I) or Formula (II): 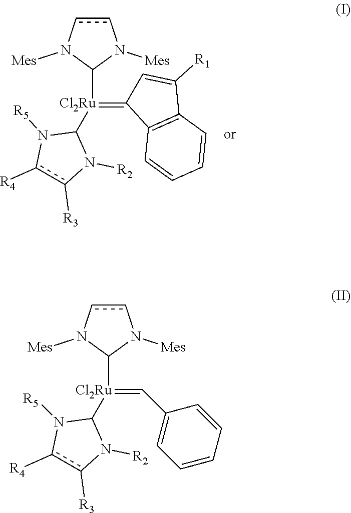
wherein:
R¹ is H, C₁₋₆alkyl, C₂₋₆alkenyl, aryl, or heteroaryl;
R² to R⁵ are, independently, H, C₁₋₆alkyl, C₁₋₆alkoxy, halo, or aryl; and
Mes is

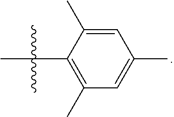 [SMILES: *c1c(C)cc(C)cc1C]

14. The method of claim 11, wherein the ruthenium catalyst is

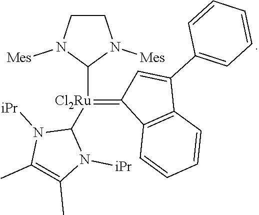

15. The method of claim 11, wherein the ruthenium catalyst is

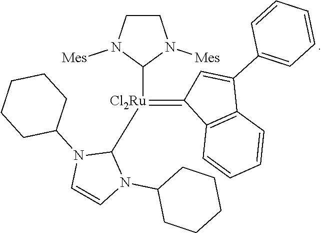

16. The method of claim 11, wherein the ruthenium catalyst is

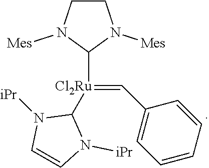

17. The method of claim 11, wherein the photo-redox catalyst is highly oxidizing.

18. The method of claim 11, wherein the photo-redox catalyst comprises Osmium.

19. The method of claim 11, wherein the photo-redox catalyst is Os(phen)₃(PF₆)₂; wherein phen is 1,10 phenanthroline.

20. The method of claim 11, wherein the photo-redox catalyst is selected from:

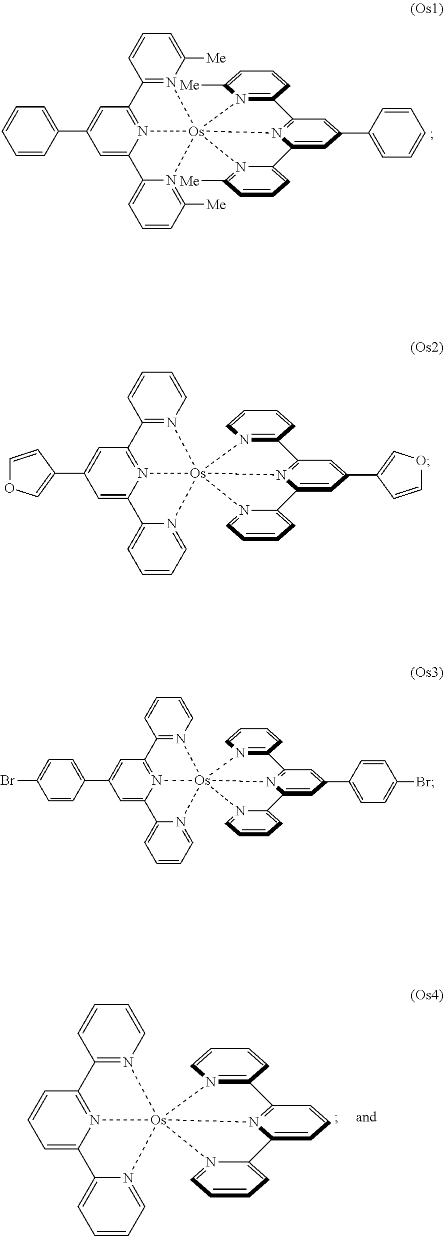

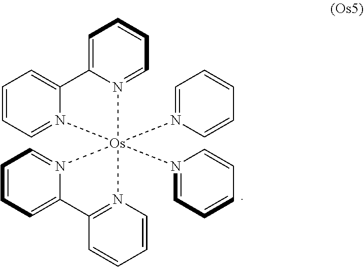

21. The method of claim 11, that is performed at a temperature of about 20 to about 30° C.

22. The method of claim 11, comprising about 0.01 to about 10 mol %, based on the mol % of the one compound or first and second compound, of the ruthenium metathesis catalyst.

23. The method of claim 11, wherein the concentration of the one compound or first and second compound is about 0.01 to about 5 M.

24. A method of spatially controlling a metathesis, comprising:
forming a mixture of a ruthenium metathesis catalyst, a photo-redox catalyst, and one or more compounds susceptible to metathesis; and
applying deep-red light or infrared light to one or more regions of the mixture so as to give rise to one or more metathesized regions and one or more un-metathesized regions.

25. The method of claim 24, wherein the infrared light is applied using a high resolution light source.

26. The method of claim 24, wherein at least one of the un-metathesized regions is covered with a photomask.

27. The method of claim 24, wherein the mixture is disposed on a substrate that is functionalized with the one or more compounds susceptible to metathesis.

28. A method of spatially controlling a metathesis, comprising:
applying deep-red light or infrared light to a ruthenium metathesis catalyst, a photo-redox catalyst, and one or more compounds susceptible to metathesis, the applying being performed so as to give rise to one or more metathesized regions,
at least one of the ruthenium metathesis catalyst and the photo-redox catalyst being linked to a substrate, the substrate optionally being stationary.

29. The method of claim 28, wherein the infrared light is applied in a predetermined pattern.
